(19) United States
(12) Patent Application Publication
(10) Pub. No.: US 2026/0306477 A1
(43) Pub. Date: Oct. 8, 2026

(54) INHALED STATINS AS BRONCHODILATORS TO IMPROVE LUNG FUNCTION IN RESPIRATORY DISEASES

(71) Applicants: The Regents of the University of California, Oakland, CA (US); Beth Israel Deaconess Medical Center, Inc., Boston, MA (US)

(72) Inventors: Amir A. Zeki, Davis, CA (US); Chandra C. Ghosh, Boston, MA (US); Ramaswamy Krishnan, Boston, MA (US)

(21) Appl. No.: 19/537,134

(22) Filed: Feb. 11, 2026

Related U.S. Application Data
(63) Continuation of application No. 17/598,568, filed on Mar. 27, 2020, now abandoned.
(60) Provisional application No. 62/826,620, filed on Mar. 29, 2019.
(60) Provisional application No. 62/906,427, filed on Sep. 26, 2019.

(51) Int. Cl. A61K 31/47; A61K 9/00; A61K 31/137; A61K 31/24; A61K 31/366; A61K 31/381; A61K 31/40; A61K 31/4025; A61K 31/439; A61K 31/44; A61K 31/4545; A61K 31/46; A61K 31/4741; A61K 31/505; A61K 31/522; A61K 31/5386; A61K 31/573; A61K 39/395; A61P 11/08

(52) U.S. Cl. CPC A61K 31/47; A61K 9/0078; A61K 31/137; A61K 31/24; A61K 31/366; A61K 31/381; A61K 31/40; A61K 31/4025; A61K 31/439; A61K 31/44; A61K 31/4545; A61K 31/46; A61K 31/4741; A61K 31/505; A61K 31/522; A61K 31/5386; A61K 31/573; A61K 39/3955; A61K 39/39566; A61P 11/08

(57) ABSTRACT

The present disclosure relates to methods for relaxing airway smooth muscle tissue, alleviating or preventing bronchospasm, and treating lung diseases by administering an HMG-CoA reductase inhibitor (statin) directly to lung tissue by inhalation. The disclosure also relates to formulations and compositions useful for the practice of methods of the disclosure.

### CROSS REFERENCE TO RELATED APPLICATIONS

This application is a continuation of U.S. application Ser. No. 17/598,568, filed Sep. 27, 2021, which is the U.S. National Stage under § 371 of International Application No. PCT/US2020/025543, filed Mar. 27, 2020, which claims the benefit under 35 U.S.C. § 119(e) of U.S. Provisional Application No. 62/826,620, filed Mar. 29, 2019, and U.S. Provisional Application No. 62/906,427, filed Sep. 26, 2019, each of which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with government support under Grant Nos. NIH/NHLBI K08 HL114882-01A1 and California Regional Primate Center Pilot Grant (P51 OD011107). The government has certain rights in the invention.

### BACKGROUND

Asthma affects nearly 20 million people in the United States and over 339 million people worldwide with symptoms of wheezing and shortness of breath due to excessive airway narrowing. See, for example, S. S. An et al., Eur Respir J (2007) 29(5):834-60; Y. Amrani et al., Int J Biochem Cell Biol (2003) 35:272-76 (http://www. globalasthmareport.org/, visited March 29, 2019). Despite widespread use of long-acting β2-agonists and high doses of inhaled corticosteroids, 55% of asthmatics experience poor symptom control leading to increased hospitalization, lost workdays, disability, and death at an estimated annual cost of ~$82 billion in 2013 (K. R. Chapman et al., Eur Respir J (2008) 31(2):320-25; S. P. Peters et al., J Allergy Clin Imunol (2007) 119:1454-61; S. Webb, Nat Publ Gr (2011) 29(10):860-63; T. Nurmagambetov et al., Ann Am Thorac Soc (2018) 15(3):348-56. This sobering reality highlights the unmet therapeutic need that persists in asthma.

Steroids act to suppress inflammatory cytokines and chemokines, block immune cell recruitment to airways and local inflammation that can sensitize airways to pro-contractile agonists, leading to airway hyperresponsiveness. In many patients with COPD and severe asthma, however, steroid insensitivity is observed even when using high doses, which leaves bronchodilator action directly on airway smooth muscle as the key mechanism to maintain lung function and provide disease control. Indeed, in COPD the standard of care often omits steroids, relying solely on anti-muscarinic or β-agonist bronchodilators to maintain disease control. The importance of bronchodilating airway smooth muscle to maintain disease control has also been recognized for a wide variety of other respiratory disorders, including cystic fibrosis (D. P. Cook et al., Am J Respir Crit Care Med (2016) 193(4):417-26); C. D. Pascoe et al., Am J Respir Cell Mol Biol (2018) doi:10.1165/rcmb.2018-0378ED). Many patients, however, remain poorly controlled despite regular use of their existing bronchodilators medications and show frequent exacerbations, indicating a need for new medicines.

Airway smooth muscle cells show phenotypic plasticity, exhibiting proliferative or contractile states. Bronchodilators target the contractile state to relax airways and provide patients with acute relief of breathlessness, improved lung function and disease control. Increases in airway smooth muscle mass have also been observed in respiratory disease, and the investigation of potential treatments to reduce airway smooth muscle proliferation and mass is also being investigated pre-clinically. However, no agents have advanced to clinical studies in humans and it remains unknown if reducing proliferation of airway smooth muscle alone would be sufficient to improve patient lung function and disease control.

During an asthma exacerbation, airway smooth muscle (ASM) contraction is the primary cause of acute bronchoconstriction (S. S. An et al., supra; R. K. Lambert et al., J Appl Physiol (1997) 83(11):140-47; P. T. Macklem, Am J Respir Crit Care Med (1996) 153:83-89). ASM mass is also substantially increased in severe and fatal asthma (L. Benayoun et al., Am J Respir Crit Care Med (2003) 167(10):1360-68; N. Carroll et al., Am Rev Respir Dis (1993) 147(2):405-10). Therefore, the therapeutic potential of agents that target ASM is, in principle, even greater in these sub-populations. However, current therapies directed at overcoming ASM contraction such as β2-agonists, muscarinic antagonists, and cysteinyl leukotriene receptor antagonists do not fully control symptoms. These therapies target receptor-mediated pathways that are complex, indirect, and susceptible to desensitization (E. J. Whalen et al., Cell (2007) 129(3):511-22). Targeting the ASM cytoskeleton is an alternative for achieving ASM relaxation and strong pre-clinical evidence has accumulated in support of targeting specific pathways such as actin, myosin, zyxin, cofilin, and Rho kinase (ROCK) mediated signaling (S. Chen et al., Am J Respir Cell Mol Biol (2014) 50:1076-83; W. T. Gerthoffer et al., Curr Opin Pharmacol (2013) 13:324-30; T. L. Lavoie et al., Proc Am Thor Soc (2009) 6:295-300; S. R. Rosner et al., PLoS One (2017) 12:e0171728; B. Lan et al., Am J Physiol Lung Cell Mol Physiol (2018) 314(5): L799-807; W. Zhang et al., J Physiol (2018) 596:3617-35). However, further development of these cytoskeletal targets has been hampered by concerns regarding their safely, specificity, and efficacy in the ASM of the asthmatic patient.

Statins are 3-hydroxy-3-methylglutaryl-coenzyme A reductase (HMG-CoA reductase) inhibitors that block the biosynthesis of mevalonate (MA) and the downstream isoprenoid lipids farnesyl-pyrophosphate (FPP) and geranylgeranyl-pyrophosphate (GGPP). At present, in the United States they are only approved for oral administration as lipid lowering agents. In asthma models, statins have pleiotropic effects such as anti-inflammatory, anti-fibrotic, anti-proliferative, and immune modulatory. Despite positive laboratory and epidemiological data, clinical trials using oral statins to ameliorate asthma symptoms have yielded conflicting and/or negative results.

While statins have been recognized as capable of reducing inflammation, it remains unclear that these anti-inflammatory effects would be beneficial to patients to improve lung function beyond other anti-inflammatory treatments that are already widely used, including steroids. Excessive smooth muscle bronchoconstriction, however, remains a daily problem for patients suffering from respiratory diseases, leading directly to airway narrowing and declinations in lung function. For patients whose disease is poorly controlled with their existing therapies, there remains a need for novel bronchodilators.

### BRIEF SUMMARY OF THE INVENTION

In one embodiment, the present disclosure provides a method for reducing airway smooth muscle contraction in a subject, the method comprising: administering a formulation to a subject having a non-inflammatory lung airway disease by inhalation, wherein the formulation comprises a therapeutically effective amount of a statin, or an isomer, enantiomer, or diastereomer thereof, and a pharmaceutically acceptable carrier. In some embodiments, the statin is selected from the group consisting of simvastatin, pitavastatin, rosuvastatin, atorvastatin, lovastatin, fluvastatin, mevastatin, cerivastatin, tenivastatin, and pravastatin, and isomers, enantiomers, and diastereomers thereof. In some embodiments, the statin is a hydrophobic statin. In some embodiments, the statin is selected from the group consisting of simvastatin, pitavastatin, rosuvastatin, and atorvastatin, and isomers, enantiomers, and diastereomers thereof. In some embodiments, the statin is selected from the group consisting of pitavastatin and isomers, enantiomers, and diastereomers thereof. In some embodiments, the statin is selected from the group consisting of pitavastatin and simvastatin. In some embodiments, the statin is pitavastatin. In some embodiments, the statin is simvastatin.

In some embodiments, the therapeutically effective amount is between about 0.005 μg and about 40 mg. In some embodiments, the therapeutically effective amount is between about 0.5 μg and about 15 mg. In some embodiments, the therapeutically effective amount is between about 1.0 μg and about 10 mg. In some embodiments, therapeutically effective amount is between about 1.0 μg and about 5 mg.

In some embodiments, the subject has been diagnosed with a lung airway disease. In some embodiments, the lung airway disease is selected from the group consisting of exercise-induced bronchospasm, exercise-induced asthma, aspirin-exacerbated respiratory disease, NSAID-exacerbated respiratory disease, paucigranulocytic asthma, obesity-associated airway hyperresponsiveness, and post-viral airway hyperresponsiveness.

In some embodiments, the lung airway disease is characterized by bronchospasm. In some embodiments, the lung disease is selected from the group consisting of post-infectious bronchospasm due to viral, bacterial, fungal, and/or mycobacterial infection; airway edema due to congestive heart failure; airway edema due to pulmonary edema; airway edema due to cardiogenic pulmonary edema; airway edema due to non-cardiogenic pulmonary edema; bronchiolitis due to airway edema; bronchiectasis due to anatomic distortions rather than inflammation; foreign body aspiration; aspiration of food, liquids, and/or gastric contents; gastro-esophageal reflux disease; lung cancer or metastatic cancer to the lung causing local edema and bronchospasm; pulmonary embolism (which can release local factors that cause wheezing due to bronchospasm); airway trauma, including surgery; anaphylaxis and anaphylactoid reactions; neurally mediated cough and/or bronchospasm; inhalation injury-associated bronchospasm; endocrine dysfunction associated bronchospasm; and paraneoplastic syndrome-associated bronchospasm.

In some embodiments, the administration is effected using a mechanical inhaler. In some embodiments, the mechanical inhaler is a metered-dose inhaler. In some embodiments, the metered-dose inhaler is a pressurized metered dose aerosol inhaler. In some embodiments, the metered-dose inhaler is a pressurized metered dose inhaler. In some embodiments, the metered-dose inhaler is a dry powder inhaler. In some embodiments, the mechanical inhaler is a nebulizer. In some embodiments, the mechanical inhaler is selected from the group consisting of: Respimat® Soft Mist™ inhaler, RespiClick® inhaler, Breezhaler® inhaler, Genuair® inhaler, and Ellipta® inhaler.

In some embodiments, the method further comprises administering one, two, or three additional therapeutic agents. In some embodiments, one, two, or three additional therapeutic agents are administered in the same formulation as the statin. In some embodiments, one, two, or three additional therapeutic agents are not administered in the same formulation as the statin. In some embodiments, at least one of the additional therapeutic agents is administered in a formulation separate from the statin. In some embodiments, the statin and one, two, or three additional therapeutic agents are administered at the same time. In some embodiments, the statin and one, two, or three additional therapeutic agents are administered at different times.

In some embodiments, the additional therapeutic agent is selected from the group consisting of β-agonists; corticosteroids; muscarinic antagonists; RhoA inhibitors; GGTase-I or -II inhibitors; ROCK1 and/or ROCK2 inhibitors; soluble epoxide hydrolase inhibitors; fatty acid amide hydrolase inhibitors; leukotriene receptor antagonists; phosphodiesterase-4 inhibitors such as roflumilast; 5-lipoxygenase inhibitors such as zileuton; mast cell stabilizers such as nedocromil; theophylline; anti-IL5 antibodies; anti-IgE antibodies; anti-IL5 receptor antibodies; anti-IL13/4 receptor antibodies; biologics such as mepolizumab, reslizumab, benralizumab, omalizumab, and dupilumab; β-agonist and muscarinic antagonist combinations, including both long-and short-acting formulations; β-agonist and corticosteroid combinations, including both long-and short-acting formulations; corticosteroids and muscarinic antagonist combinations, including both long-and short-acting formulations; and β-agonist, corticosteroid, and muscarinic antagonist combinations, including both long-and short-acting formulations. In some embodiments, the additional therapeutic agent is a β-agonist is selected from the group consisting of albuterol, aformoterol, formoterol, salmeterol, indacaterol, levalbuterol, salbutamol, terbutaline, olodaterol, vilanterol, isoxsuprine, mabuterol, zilpaterol, bambuterol, clenbuterol, formoterol, salmeterol, abediterol, and carmoterol, buphenine, bopexamine, epinephrine, fenoterol, isoetarine, isoproterenol, orciprenaline, levoalbutamol, pirbuterol, procaterol, ritodrine, arbutamine, befunolol, bromoacetylalprenololmenthane, broxaterol, cimaterol, cirazoline, etilefrine, hexoprenaline, higenamine, methoxyphenamine, oxyfedrine, ractopamine, reproterol, rimiterol, tretoquinol, tulobuterol, zilpaterol, and zintero.

In some embodiments, the additional therapeutic agent is a corticosteroid selected from the group consisting of beclomethasone, fluticasone, budesonide, mometasone, flunisolide, alclometasone, beclometasone, betamethasone, clobetasol, clobetasone, clocortolone, desoximetasone, dexamethasone, diflorasone, difluocortolone, flurclorolone, flumetasone, fluocortin, fluocortolone, fluprednidene, fluticasone, fluticasone furoate, halometasone, meprednisone, mometasone, mometasone furoate, paramethasone, prednylidene, rimexolone, ulobetasol, amcinonide, ciclesonide, deflazacort, desonide, formocortal, fluclorolone acetonide, fludroxycortide, fluocinolone acetonide, fluocinonide, halcinonide, and triamcinolone acetonide. In some embodiments, the additional therapeutic agent is a muscarinic antagonist selected from the group consisting of ipratropium bromide, tiotropium, glycopyrrolate, glycopyrronium bromide, revefenacin, umeclidinium bromide, aclidinium, trospium chloride, oxitropium bromide, oxybutynin, tolterodine, solifenacin, fesoterodine, and darifenacin. In some embodiments, the additional therapeutic agent is a ROCK inhibitor selected from the group consisting of: fasudil, ripasudil, netarsudil, RKI-1447, Y-27632, Y-30141, and GSK429286A. In some embodiments, the additional therapeutic agent is the RhoA inhibitor rhosin.

In some embodiments, one, two, or three additional therapeutic agents are potentiated by the statin. In some embodiments, one, two, or three additional therapeutic agents are administered at a sub-therapeutic dose.

In some embodiments, the pharmaceutically acceptable carrier comprises a component selected from the group consisting of: monosaccharides, disaccharides, oligo- and polysaccharides, polyalcohols, cyclodextrins, DexSol, amino acids, salts, and mixtures thereof.

In some embodiments, the component comprises a monosaccharide selected from the group consisting of glucose, fructose, and arabinose. In some embodiments, the component comprises a disaccharide selected from the group consisting of lactose, saccharose, maltose, and trehalose. In some embodiments, the component comprises an oligo-or polysaccharide selected from the group consisting of dextrans, dextrins, maltodextrin, starch, and cellulose. In some embodiments, the component comprises a polyalcohol selected from the group consisting of sorbitol, mannitol, and xylitol. In some embodiments, the component comprises a cyclodextrin selected from the group consisting of α-cyclodextrin, β-cyclodextrin, χ-cyclodextrin, methyl-β-cyclodextrin, and hydroxypropyl-β-cyclodextrin, captisol, and sulfobutyl-β-cyclodextrin. In some embodiments, the component comprises arginine or arginine hydrochloride. In some embodiments, the component comprises a salt selected from the group consisting of sodium chloride, potassium chloride, sodium bromide, and calcium carbonate.

In another embodiment, the present disclosure provides a method for treating bronchospasm in a subject, by administering a formulation by inhalation to a subject in need thereof, where the formulation contains an effective amount of a statin, or an isomer, enantiomer, or diastereomer thereof, and a pharmaceutically acceptable carrier. In some embodiments, the statin is selected from the group consisting of simvastatin, pitavastatin, rosuvastatin, atorvastatin, lovastatin, fluvastatin, mevastatin, cerivastatin, tenivastatin, and pravastatin, and isomers, enantiomers, and diastereomers thereof. In some embodiments, the statin is selected from the group consisting of simvastatin, pitavastatin, rosuvastatin, and atorvastatin, and isomers, enantiomers, and diastereomers thereof. In some embodiments, the statin is selected from the group consisting of simvastatin and pitavastatin. In some embodiments, the statin is selected from the group consisting of pitavastatin and isomers, enantiomers, and diastereomers thereof. In some embodiments, the statin is comprises pitavastatin.

In some embodiments, the subject has been diagnosed with a lung airway disease. In some embodiments, the lung airway disease is selected from the group consisting of asthma; exercise-induced bronchoconstriction (or exercise-induced asthma); COPD which can include emphysema, chronic bronchitis, and/or alpha-1 antitrypsin deficiency (AATD); ACOS; cystic fibrosis; and bronchiectasis. In some embodiments, the lung airway disease is a non-inflammatory lung airway disease. In some embodiments, the lung airway disease is selected from the group consisting of exercise-induced bronchospasm, exercise-induced asthma, aspirin-exacerbated respiratory disease, NSAID-exacerbated respiratory disease, paucigranulocytic asthma, obesity-associated airway hyperresponsiveness, and post-viral airway hyperresponsiveness. In some embodiments, the lung airway disease is characterized by airway smooth muscle contraction. In some embodiments, the lung disease is selected from the group consisting of post-infectious bronchospasm due to viral, bacterial, fungal, and/or mycobacterial infection; airway edema due to congestive heart failure; airway edema due to pulmonary edema; airway edema due to cardiogenic pulmonary edema; airway edema due to non-cardiogenic pulmonary edema; bronchiolitis due to airway edema; bronchiectasis due to anatomic distortions rather than inflammation; foreign body aspiration; aspiration of food, liquids, and/or gastric contents; gastro-esophageal reflux disease; lung cancer or metastatic cancer to the lung causing local edema and bronchospasm; pulmonary embolism (which can release local factors that cause wheezing due to bronchospasm); airway trauma, including surgery; anaphylaxis and anaphylactoid reactions; neurally mediated cough and/or bronchospasm; inhalation injury-associated bronchospasm; endocrine dysfunction associated bronchospasm; and paraneoplastic syndrome-associated bronchospasm.

In some embodiments, the lung airway disease is characterized by bronchospasm. In some embodiments, the administration effected using a mechanical inhaler. In some embodiments, the mechanical inhaler is a metered-dose inhaler. In some embodiments, the metered-dose inhaler is a pressurized aerosol metered dose inhaler. In some embodiments, the metered-dose inhaler is a dry powder inhaler. In some embodiments, the mechanical inhaler is a nebulizer. In some embodiments, the mechanical inhaler is selected from the group consisting of: Respimat® Soft Mist™ inhaler, RespiClick® inhaler, Breezhaler® inhaler, Genuair® inhaler, PulmoSphere carrier inhaler, and Ellipta® inhaler.

In some embodiments, the formulation further comprises one, two, or three additional therapeutic agents. In some embodiments, at least one of the additional therapeutic agents is potentiated by the statin. In some embodiments, an additional therapeutic agent is administered at a sub-therapeutic dose. In some embodiments, one, two, or three additional therapeutic agents are administered at a sub-therapeutic dose. In some embodiments, the additional therapeutic agent is selected from the group consisting of β-agonists; corticosteroids; muscarinic antagonists; RhoA inhibitors; GGTase-I or -II inhibitors; ROCK1 and/or ROCK2 inhibitors; soluble epoxide hydrolase inhibitors; fatty acid amide hydrolase inhibitors; leukotriene receptor antagonists; phosphodiesterase-4 inhibitors such as roflumilast; 5-lipoxygenase inhibitors such as zileuton; mast cell stabilizers such as nedocromil; theophylline; anti-IL5 antibodies; anti-IgE antibodies; anti-IL5 receptor antibodies; anti-IL13/4 receptor antibodies; biologics such as mepolizumab, reslizumab, benralizumab, omalizumab, and dupilumab; β-agonist and muscarinic antagonist combinations, including both long-and short-acting formulations; β-agonist and corticosteroid combinations, including both long-and short-acting formulations; corticosteroids and muscarinic antagonist combinations, including both long-and short-acting formulations; and β-agonist, corticosteroid, and muscarinic antagonist combinations, including both long-and short-acting formulations.

In some embodiments, the additional therapeutic agent is a β-agonist selected from the group consisting of: arformoterol, buphenine, clenbuterol, bopexamine, epinephrine, fenoterol, formoterol, isoetarine, isoproterenol, orciprenaline, levoalbutamol, levalbuterol, pirbuterol, procaterol, ritodrine, albuterol, salmeterol, terbutaline, arbutamine, befunolol, bromoacetylalprenololmenthane, broxaterol, cimaterol, cirazoline, etilefrine, hexoprenaline, higenamine, isoxsuprine, mabuterol, methoxyphenamine, oxyfedrine, ractopamine, reproterol, rimiterol, tretoquinol, tulobuterol, zilpaterol, and zinterol. In some embodiments, the additional therapeutic agent is a ROCK inhibitor selected from the group consisting of: fasudil, ripasudil, netarsudil, RKI-1447, Y-27632, Y-30141, and GSK429286A. In some embodiments, the second therapeutic agent is the RhoA inhibitor rhosin.

In some embodiments, the pharmaceutically acceptable carrier comprises a component selected from the group consisting of: monosaccharides, disaccharides, oligo-and polysaccharides, polyalcohols, cyclodextrins, amino acids, salts, and mixtures thereof. In some embodiments, the component comprises a monosaccharide selected from the group consisting of glucose, fructose, and arabinose. In some embodiments, the component comprises a disaccharide selected from the group consisting of lactose, saccharose, maltose, and trehalose. In some embodiments, the component comprises an oligo-or polysaccharide selected from the group consisting of dextrans, dextrins, maltodextrin, starch, and cellulose. In some embodiments, the component comprises a polyalcohol selected from the group consisting of sorbitol, mannitol, and xylitol. In some embodiments, the component comprises a cyclodextrin selected from the group consisting of α-cyclodextrin, β-cyclodextrin, χ-cyclodextrin, methyl-β-cyclodextrin, and hydroxypropyl-β-cyclodextrin. In some embodiments, the component comprises arginine or arginine hydrochloride. In some embodiments, the component comprises a salt selected from the group consisting of sodium chloride, potassium chloride, sodium bromide, and calcium carbonate.

In another embodiment, the present disclosure provides a pharmaceutical formulation for the treatment of a lung airway disease, the composition comprising a therapeutically effective amount of a statin, or an isomer, enantiomer, or diastereomer thereof, and a pharmaceutically acceptable carrier suitable for administration by inhalation. In some embodiments, the statin is selected from the group consisting of simvastatin, pitavastatin, rosuvastatin, atorvastatin, lovastatin, fluvastatin, mevastatin, cerivastatin, tenivastatin, and pravastatin, and isomers, enantiomers, and diastereomers thereof. In some embodiments, the statin is selected from the group consisting of simvastatin, pitavastatin, rosuvastatin, and atorvastatin, and isomers, enantiomers, and diastereomers thereof. In some embodiments, the statin is selected from the group consisting of pitavastatin and simvastatin, and isomers, enantiomers, and diastereomers thereof. In some embodiments, the statin is pitavastatin. In some embodiments, the statin is simvastatin.

In some embodiments, the effective amount is between about 0.005 mg and about 80 mg. In some embodiments, the effective amount is between about 0.5 mg and about 15 mg. In some embodiments, the effective amount is between about 1.0 mg and about 10 mg. In some embodiments, the effective amount is between about 1.0 mg and about 5 mg.

In some embodiments, the formulation further comprises one, two, or three additional therapeutic agents. In some embodiments, at least one of the additional therapeutic agents is potentiated by the statin. In some embodiments, an additional therapeutic agent is administered at a sub-therapeutic dose. In some embodiments, one, two, or three additional therapeutic agents are administered at a sub-therapeutic dose. In some embodiments, the formulation further comprises an additional therapeutic agent selected from the group consisting of β-agonists; corticosteroids; muscarinic antagonists; RhoA inhibitors; GGTase-I or -II inhibitors; ROCK1 and/or ROCK2 inhibitors; soluble epoxide hydrolase inhibitors; fatty acid amide hydrolase inhibitors; leukotriene receptor antagonists; phosphodiesterase-4 inhibitors such as roflumilast; 5-lipoxygenase inhibitors such as zileuton; mast cell stabilizers such as nedocromil; theophylline; anti-IL5 antibodies; anti-IgE antibodies; anti-IL5 receptor antibodies; anti-IL13/4 receptor antibodies; biologics such as mepolizumab, reslizumab, benralizumab, omalizumab, and dupilumab; β-agonist and muscarinic antagonist combinations, including both long-and short-acting formulations; β-agonist and corticosteroid combinations, including both long-and short-acting formulations; corticosteroids and muscarinic antagonist combinations, including both long- and short-acting formulations; and β-agonist, corticosteroid, and muscarinic antagonist combinations, including both long- and short-acting formulations.

In some embodiments, the additional therapeutic agent is a β-agonist selected from the group consisting of: arformoterol, buphenine, clenbuterol, levalbuterol, bopexamine, epinephrine, fenoterol, formoterol, isoetarine, isoproterenol, orciprenaline, levoalbutamol, pirbuterol, procaterol, ritodrine, albuterol, salmeterol, terbutaline, arbutamine, befunolol, bromoacetylalprenololmenthane, broxaterol, cimaterol, cirazoline, etilefrine, hexoprenaline, higenamine, isoxsuprine, mabuterol, methoxyphenamine, oxyfedrine, ractopamine, reproterol, rimiterol, tretoquinol, tulobuterol, zilpaterol, and zinterol. In some embodiments, the additional therapeutic agent is a ROCK inhibitor selected from the group consisting of: fasudil, ripasudil, netarsudil, RKI-1447, Y-27632, Y-30141, and GSK429286A. In some embodiments, the additional therapeutic agent is the RhoA inhibitor rhosin.

In some embodiments, the pharmaceutically acceptable carrier comprises a component selected from the group consisting of: monosaccharides, disaccharides, oligo-and polysaccharides, polyalcohols, cyclodextrins, amino acids, salts, and mixtures thereof. In some embodiments, the component comprises a monosaccharide selected from the group consisting of glucose, fructose, and arabinose. In some embodiments, the component comprises a disaccharide selected from the group consisting of lactose, saccharose, maltose, and trehalose. In some embodiments, the component comprises an oligo- or polysaccharide selected from the group consisting of dextrans, dextrins, maltodextrin, starch, and cellulose. In some embodiments, the component comprises a polyalcohol selected from the group consisting of sorbitol, mannitol, and xylitol. In some embodiments, the component comprises a cyclodextrin selected from the group consisting of α-cyclodextrin, β-cyclodextrin, χ-cyclodextrin, methyl-β-cyclodextrin, and hydroxypropyl-β-cyclodextrin. In some embodiments, the component comprises arginine or arginine hydrochloride. In some embodiments, the component comprises a salt selected from the group consisting of sodium chloride, potassium chloride, sodium bromide, and calcium carbonate.

In another embodiment, the present disclosure provides a pre-filled inhalation device, for treating a lung airway disease in a subject, the device comprising a delivery device for delivering a therapeutic dose of a formulation to the lung airways of a subject in need thereof; and a pharmaceutically acceptable formulation as described herein. In some embodiments, the device comprises a pressurized inhaler, a metered dose inhaler, a dry powder inhaler, or a nebulizer.

In some embodiments, the device contains multiple therapeutic doses. In some embodiments, the delivery device is a metered-dose inhaler. In some embodiments, the metered-dose inhaler is a pressurized aerosol inhaler. In some embodiments, the metered-dose inhaler is a dry powder inhaler. In some embodiments, the delivery device is a nebulizer. In some embodiments, the delivery device is selected from the group consisting of: Respimat® Soft Mist™ inhaler, RespiClick® inhaler, Breezhaler® inhaler, Genuair® inhaler, PulmoSphere carrier inhaler, and Ellipta® inhaler.

In another embodiment, the present disclosure provides a pre-filled cartridge for use with an inhaler, comprising a container comprising linking means for attaching the container to an inhaler device; and a pharmaceutically acceptable formulation as described herein. In some embodiments, inhaler device further comprises a pharmaceutically acceptable propellant.

In another embodiment, the present disclosure provides any of the methods above, wherein the therapeutically effective amount is effective for the maintenance of lung function; for the reduction of asthma exacerbations; for reduction of the subject's need for corticosteroids; for reduction of bronchoconstriction and mucus accumulation in the subject; or for potentiation of breathing-induced bronchodilation. In some embodiments, the therapeutically effective amount is effective for the maintenance of lung function; for the reduction of asthma exacerbations; for reduction of bronchoconstriction and mucus accumulation in the subject; or for potentiation of breathing-induced bronchodilation

In another embodiment, the present disclosure provides a method for reducing airway hyperresponsiveness (AHR) or ASM hypercontraction in a subject, the method comprising administering a formulation of the disclosure to a subject in need thereof by inhalation, wherein the therapeutically effective amount is effective to reduce AHR or ASM hypercontraction in the subject.

In another embodiment, the present disclosure provides a method for increasing stretch-induced airway smooth muscle (ASM) relaxation in a subject, the method comprising administering a formulation of the disclosure to a subject in need thereof by inhalation, wherein the therapeutically effective amount is effective to increase stretch-induced ASM relaxation in the subject.

In another embodiment, the present disclosure provides a method for potentiating the bronchodilatory effect of a β2-agonist on ASM, comprising contacting the ASM with a potentiating amount of a statin; and contacting the ASM with a potentiating amount of a β2-agonist, wherein the resulting potentiated effect comprises ASM relaxation. In some embodiments, the ASM is contacted with the β2-agonist between about 2 hours and about 24 hours after contact with the statin. In some embodiments, the ASM is in a human subject in need of ASM relaxation. In some embodiments, the statin and the β2-agonist are administered by inhalation. In some embodiments, the potentiated effect reduces ASM contraction by at least about 10% more than the sum of the ASM contraction reduction percentage due to the β2-agonist alone and the ASM contraction reduction percentage due to the statin alone. In some embodiments, the potentiated effect reduces ASM contraction by about 10% to about 30% more than the ASM contraction reduction percentage in the absence of statin.

In some embodiments, the administration is effected using a mechanical inhaler. In some embodiments, the mechanical inhaler is a metered-dose inhaler. In some embodiments, the metered-dose inhaler is a pressurized aerosol inhaler. In some embodiments, the metered-dose inhaler is a dry powder inhaler. In some embodiments, the mechanical inhaler is a nebulizer. In some embodiments, the mechanical inhaler is selected from the group consisting of: Respimat® Soft Mist™ inhaler, RespiClick® inhaler, Breezhaler® inhaler, Genuair® inhaler, and Ellipta® inhaler.

In another embodiment, the present disclosure provides a method for treating the symptoms of an interstitial lung disease, the method comprising administering a formulation of the disclosure to a subject in need thereof by inhalation, wherein the interstitial lung disease causes an airway symptom selected from the group consisting of ASM contraction, ASM hyperproliferation or thickening, bronchospasm, bronchoconstriction, airway mucus accumulation, or ASM release of an inflammatory mediator, wherein therapeutically effective amount is effective to reduce the severity of the symptom by at least 10%.

In another embodiment, the present disclosure provides a method for treating a lung airway disease in a subject, by administering a formulation by inhalation to a subject having a lung disease, wherein the formulation comprises a pharmaceutically acceptable carrier and a therapeutically effective amount of a statin, or an isomer, enantiomer, or diastereomer thereof; and administering one, two, or three additional therapeutic agents. In some embodiments, the one, two, or three additional therapeutic agents are selected from β-agonists; corticosteroids; muscarinic antagonists; RhoA inhibitors; GGTase-I or -II inhibitors; ROCK1 and/or ROCK2 inhibitors; soluble epoxide hydrolase inhibitors; fatty acid amide hydrolase inhibitors; leukotriene receptor antagonists; phosphodiesterase-4 inhibitors such as roflumilast; 5-lipoxygenase inhibitors such as zileuton; mast cell stabilizers such as nedocromil; theophylline; anti-IL5 antibodies or antibody derivatives; anti-IgE antibodies or antibody derivatives; anti-IL5 receptor antibodies or antibody derivatives; anti-IL13/4 receptor antibodies or antibody derivatives; biologics such as mepolizumab, reslizumab, benralizumab, omalizumab, and dupilumab; β-agonist and muscarinic antagonist combinations, including both long- and short-acting formulations; β-agonist and corticosteroid combinations, including both long- and short-acting formulations; corticosteroids and muscarinic antagonist combinations, including both long- and short-acting formulations; and β-agonist, corticosteroid, and muscarinic antagonist combinations, including both long- and short-acting formulation.

In some embodiments, the additional therapeutic agent is a β-agonist is selected from the group consisting of albuterol, aformoterol, formoterol, salmeterol, indacaterol, levalbuterol, salbutamol, terbutaline, olodaterol, vilanterol, isoxsuprine, mabuterol, zilpaterol, bambuterol, clenbuterol, formoterol, salmeterol, abediterol, and carmoterol, buphenine, bopexamine, epinephrine, fenoterol, isoetarine, isoproterenol, orciprenaline, levoalbutamol, pirbuterol, procaterol, ritodrine, arbutamine, befunolol, bromoacetylalprenololmenthane, broxaterol, cimaterol, cirazoline, etilefrine, hexoprenaline, higenamine, methoxyphenamine, oxyfedrine, ractopamine, reproterol, rimiterol, tretoquinol, tulobuterol, zilpaterol, and zintero.

In some embodiments, the additional therapeutic agent is a corticosteroid selected from the group consisting of beclomethasone, fluticasone, budesonide, mometasone, flunisolide, alclometasone, beclometasone, betamethasone, clobetasol, clobetasone, clocortolone, desoximetasone, dexamethasone, diflorasone, difluocortolone, flurclorolone, flumetasone, fluocortin, fluocortolone, fluprednidene, fluticasone, fluticasone furoate, halometasone, meprednisone, mometasone, mometasone furoate, paramethasone, prednylidene, rimexolone, ulobetasol, amcinonide, ciclesonide, deflazacort, desonide, formocortal, fluclorolone acetonide, fludroxycortide, fluocinolone acetonide, fluocinonide, halcinonide, and triamcinolone acetonide. In some embodiments, the additional therapeutic agent is a muscarinic antagonist selected from the group consisting of ipratropium bromide, tiotropium, glycopyrrolate, glycopyrronium bromide, revefenacin, umeclidinium bromide, aclidinium, trospium chloride, oxitropium bromide, oxybutynin, tolterodine, solifenacin, fesoterodine, and darifenacin. In some embodiments, the additional therapeutic agent is a ROCK inhibitor selected from the group consisting of: fasudil, ripasudil, netarsudil, RKI-1447, Y-27632, Y-30141, and GSK429286A. In some embodiments, the additional therapeutic agent is the RhoA inhibitor rhosin.

In some embodiments, the additional therapeutic agent is a β-agonist, a corticosteroid, a muscarinic antagonist, or any combination thereof. In some embodiments, one, two, or three additional therapeutic agents are potentiated by the statin. In some embodiments, one, two, or three additional therapeutic agents are administered at a sub-therapeutic dose.

In some embodiments, the statin is selected from the group consisting of simvastatin, pitavastatin, rosuvastatin, atorvastatin, lovastatin, fluvastatin, mevastatin, cerivastatin, tenivastatin, and pravastatin, and isomers, enantiomers, and diastereomers thereof. In some embodiments, the statin is a hydrophobic statin. In some embodiments, the statin is selected from the group consisting of simvastatin, pitavastatin, rosuvastatin, and atorvastatin, and isomers, enantiomers, and diastereomers thereof. In some embodiments, the statin is selected from the group consisting of pitavastatin and isomers, enantiomers, and diastereomers thereof. In some embodiments, the statin is selected from the group consisting of pitavastatin and simvastatin. In some embodiments, the statin is pitavastatin. In some embodiments, the statin is simvastatin.

In some embodiments, the therapeutically effective amount is between about 0.005 μg and about 40 mg. In some embodiments, the therapeutically effective amount is between about 0.5 μg and about 15 mg. In some embodiments, the therapeutically effective amount is between about 1.0 μg and about 10 mg. In some embodiments, therapeutically effective amount is between about 1.0 μg and about 5 mg.

In some embodiments, the subject has been diagnosed with a lung airway disease. In some embodiments, the lung airway disease is asthma; exercise-induced bronchoconstriction; COPD; emphysema; chronic bronchitis; alpha-1 antitrypsin deficiency (AATD); ACOS; cystic fibrosis; bronchiectasis; exercise-induced bronchospasm, exercise-induced asthma, aspirin-exacerbated respiratory disease, NSAID-exacerbated respiratory disease, paucigranulocytic asthma, obesity-associated airway hyperresponsiveness, post-viral airway hyperresponsiveness; post-infectious bronchospasm due to viral, bacterial, fungal, and/or mycobacterial infection; airway edema due to congestive heart failure; airway edema due to pulmonary edema; airway edema due to cardiogenic pulmonary edema; airway edema due to non-cardiogenic pulmonary edema; bronchiolitis due to airway edema; bronchiectasis due to anatomic distortions rather than inflammation; foreign body aspiration; aspiration of food, liquids, and/or gastric contents; gastro-esophageal reflux disease; lung cancer or metastatic cancer to the lung causing local edema and bronchospasm; pulmonary embolism; airway trauma; surgery; anaphylaxis and anaphylactoid reactions; neurally mediated cough and/or bronchospasm; inhalation injury-associated bronchospasm; endocrine dysfunction associated bronchospasm; and paraneoplastic syndrome-associated bronchospasm. In some embodiments, the lung disease is selected from the group consisting of exercise-induced bronchospasm, exercise-induced asthma, aspirin-exacerbated respiratory disease, NSAID-exacerbated respiratory disease, paucigranulocytic asthma, obesity-associated airway hyperresponsiveness, and post-viral airway hyperresponsiveness.

In some embodiments, the lung airway disease is characterized by bronchospasm. In some embodiments, the lung disease is selected from the group consisting of post-infectious bronchospasm due to viral, bacterial, fungal, and/or mycobacterial infection; airway edema due to congestive heart failure; airway edema due to pulmonary edema; airway edema due to cardiogenic pulmonary edema; airway edema due to non-cardiogenic pulmonary edema; bronchiolitis due to airway edema; bronchiectasis due to anatomic distortions rather than inflammation; foreign body aspiration; aspiration of food, liquids, and/or gastric contents; gastro-esophageal reflux disease; lung cancer or metastatic cancer to the lung causing local edema and bronchospasm; pulmonary embolism (which can release local factors that cause wheezing due to bronchospasm); airway trauma, including surgery; anaphylaxis and anaphylactoid reactions; neurally mediated cough and/or bronchospasm; inhalation injury-associated bronchospasm; endocrine dysfunction associated bronchospasm; and paraneoplastic syndrome-associated bronchospasm.

In some embodiments, the administration is effected using a mechanical inhaler. In some embodiments, the mechanical inhaler is a metered-dose inhaler. In some embodiments, the metered-dose inhaler is a pressurized metered dose aerosol inhaler. In some embodiments, the metered-dose inhaler is a pressurized metered dose inhaler. In some embodiments, the metered-dose inhaler is a dry powder inhaler. In some embodiments, the mechanical inhaler is a nebulizer. In some embodiments, the mechanical inhaler is selected from the group consisting of: Respimat® Soft Mist™ inhaler, RespiClick& inhaler, Breezhaler® inhaler, Genuair® inhaler, and Ellipta® inhaler.

In an embodiment, the disclosure provides a method for reducing future symptoms caused by an event that has already occurred or is expected to be experienced in the future, the method comprising administering to a subject at risk of experiencing the future symptoms a formulation of the disclosure. In some embodiments, the method is wherein the future symptom is bronchospasm caused by post-infectious bronchospasm due to viral, bacterial, fungal, and/or mycobacterial infection; airway edema due to congestive heart failure; airway edema due to pulmonary edema; airway edema due to cardiogenic pulmonary edema; airway edema due to non-cardiogenic pulmonary edema; bronchiolitis due to airway edema; bronchiectasis due to anatomic distortions rather than inflammation; foreign body aspiration; aspiration of food, liquids, and/or gastric contents; gastro-esophageal reflux disease; lung cancer or metastatic cancer to the lung causing local edema and bronchospasm; pulmonary embolism; airway trauma; surgery; anaphylaxis and anaphylactoid reactions; neurally mediated cough and/or bronchospasm; inhalation injury-associated bronchospasm; endocrine dysfunction associated bronchospasm; or paraneoplastic syndrome-associated bronchospasm.

### BRIEF DESCRIPTION OF THE DRAWINGS

FIGS. 1A through 1D show that differential statin effects on the inhibition of basal ASM cell contraction occur by a mevalonate (MA)-dependent mechanism. ASMs were treated for 24 hours with 1 μM of statins. P-values: **p<0.01, ***p<0.001. NT: not treated; Pra: pravastatin;

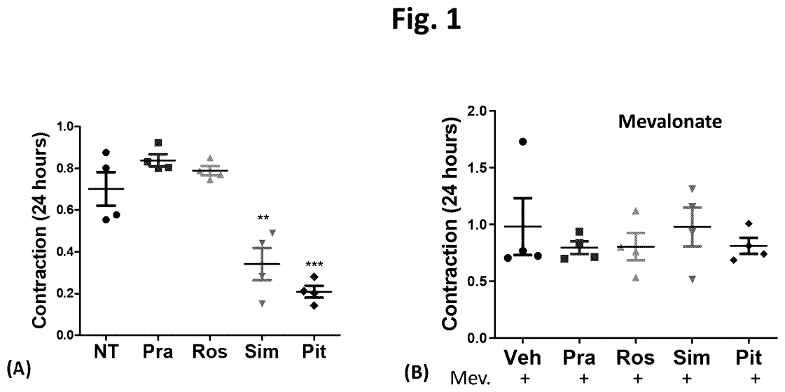

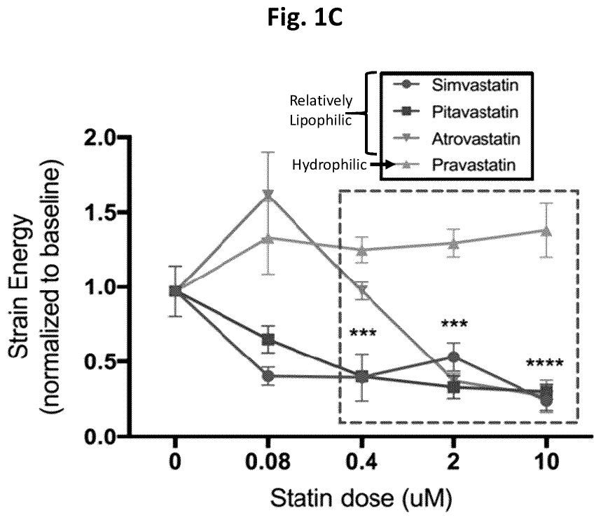

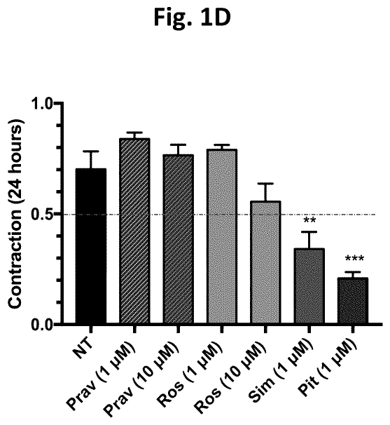

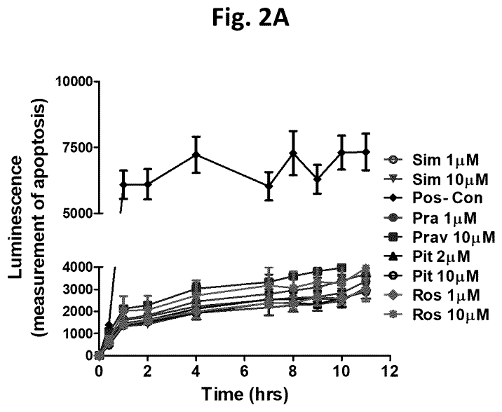

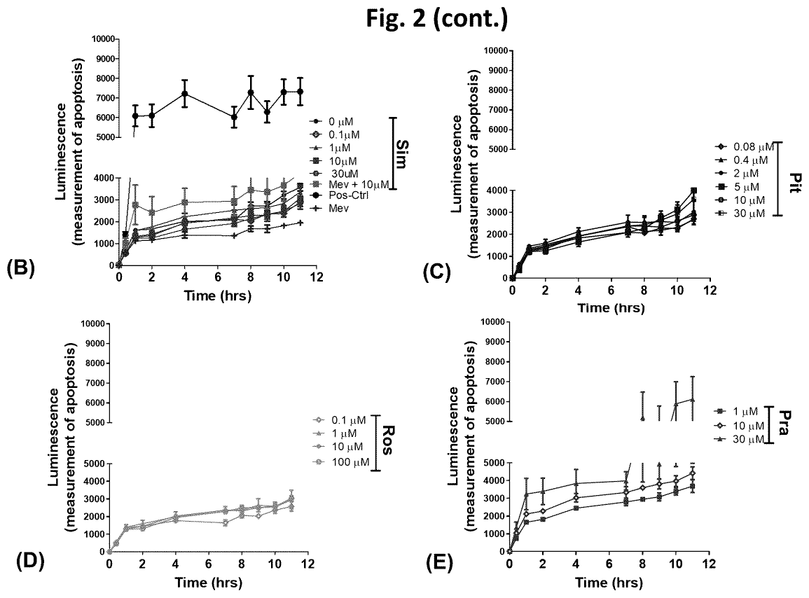

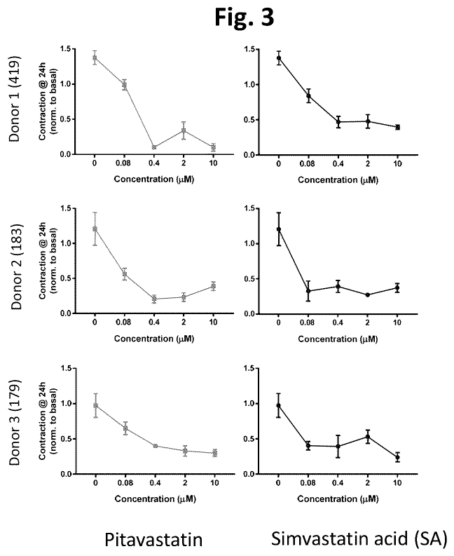

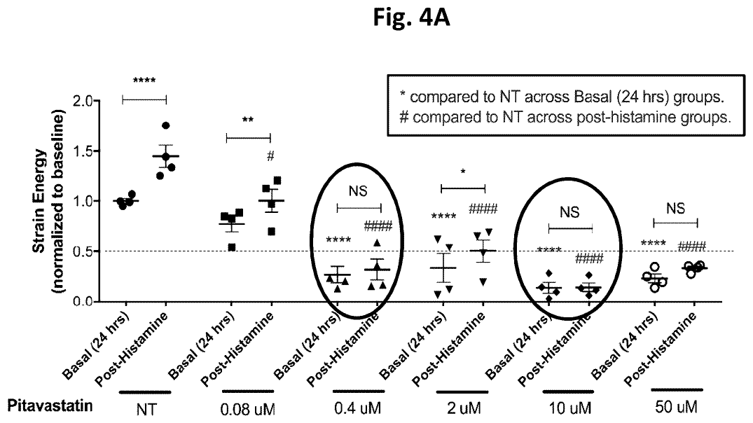

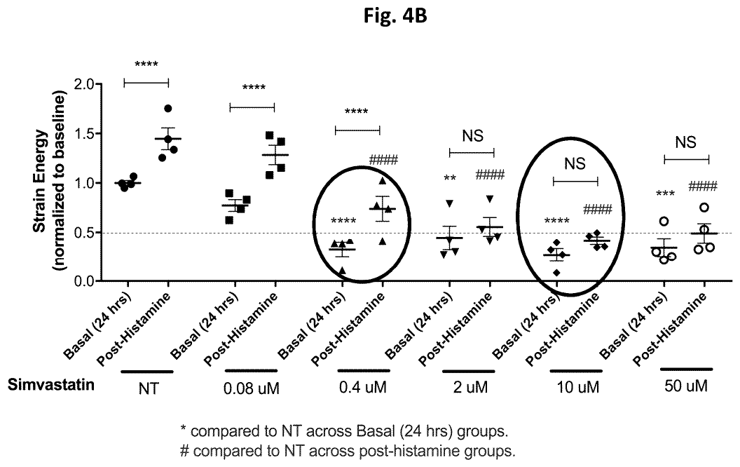

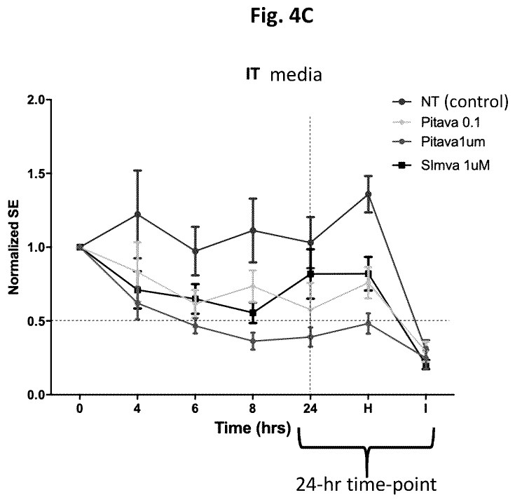

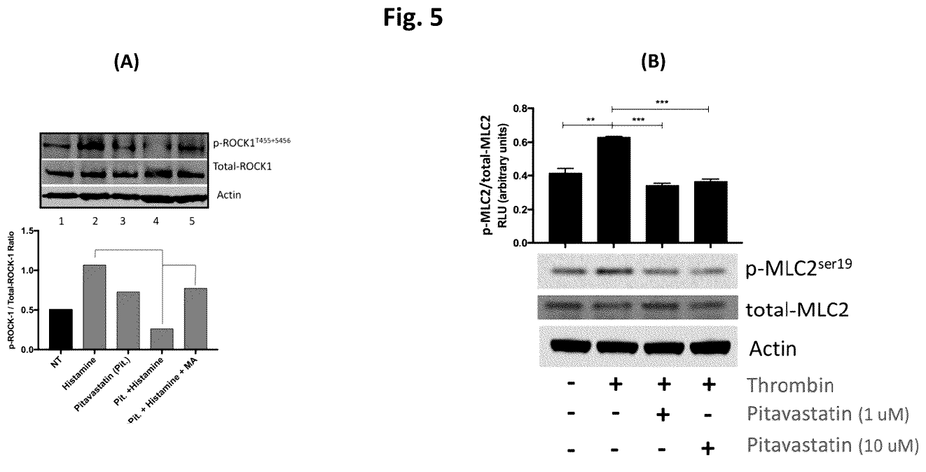

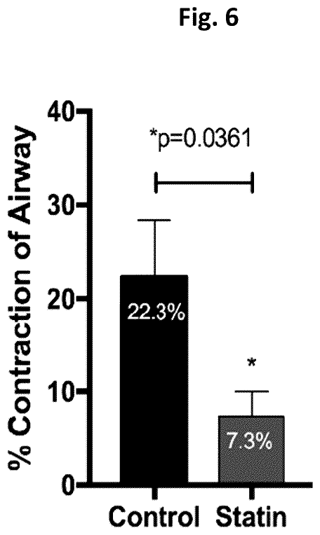

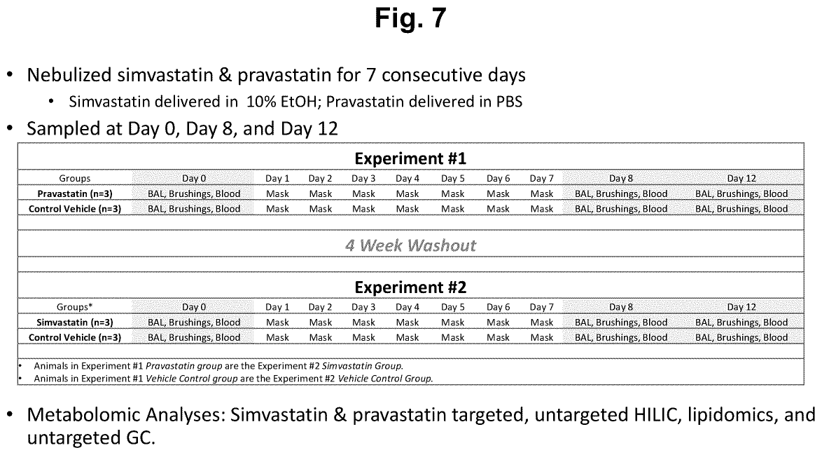

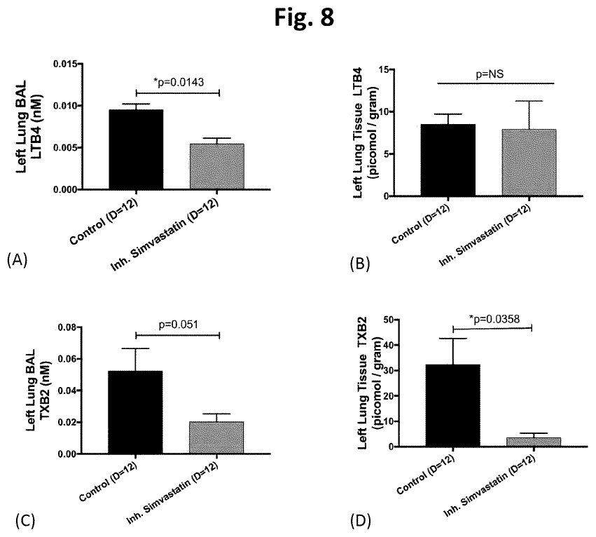

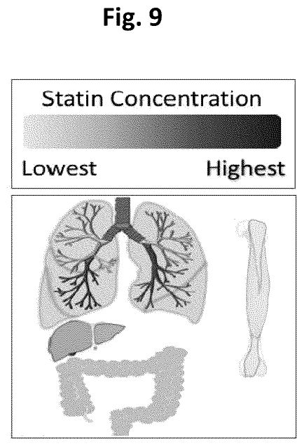

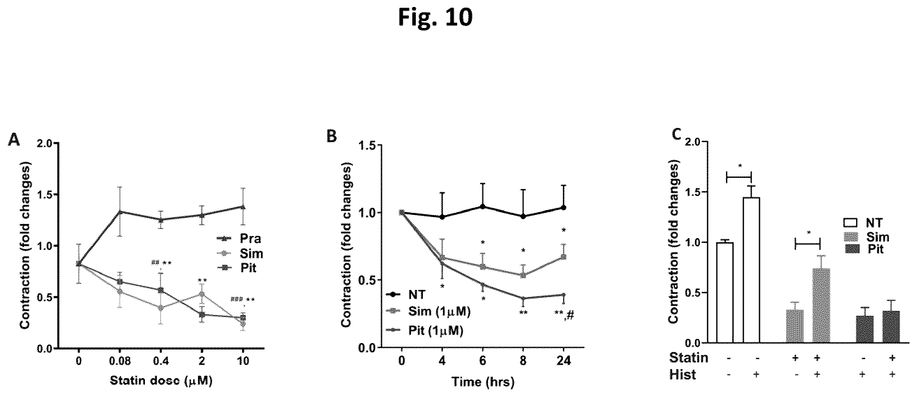

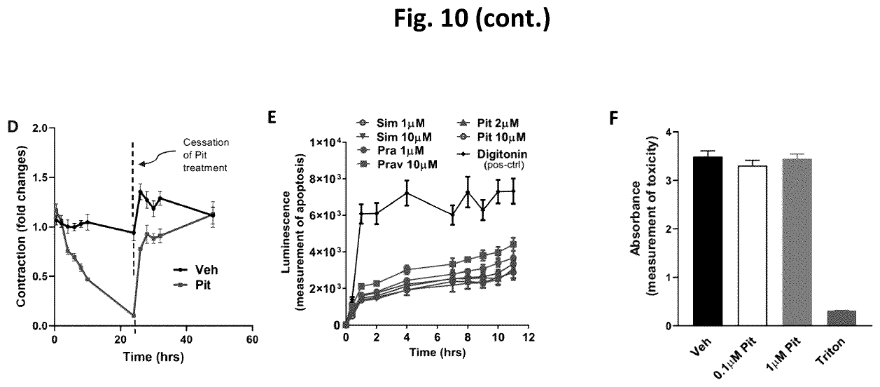

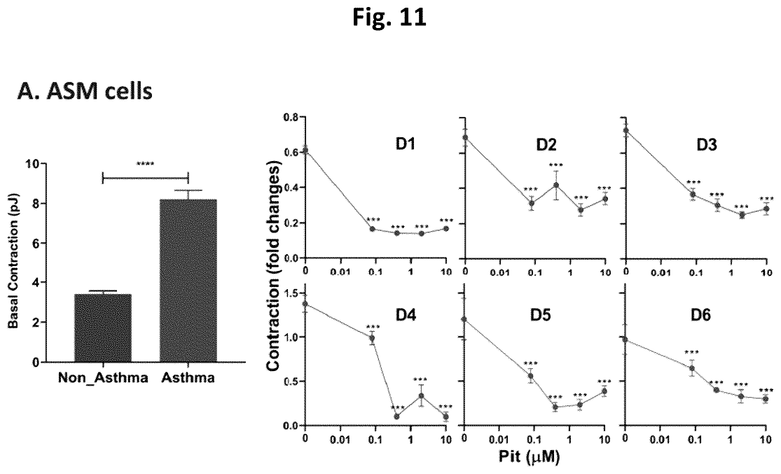

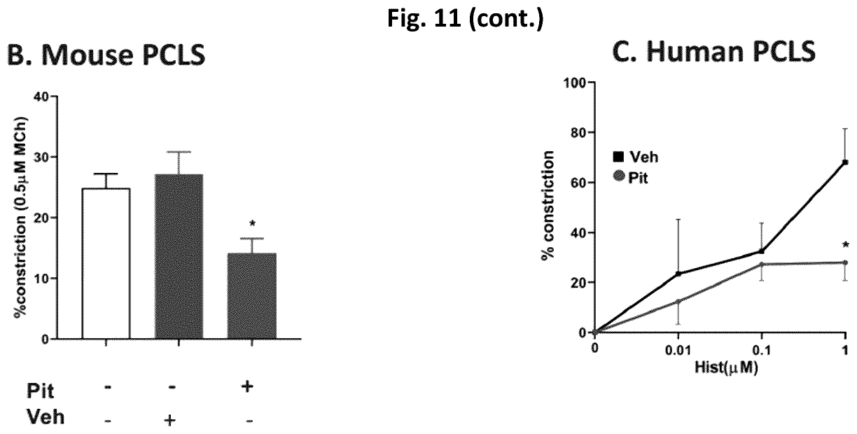

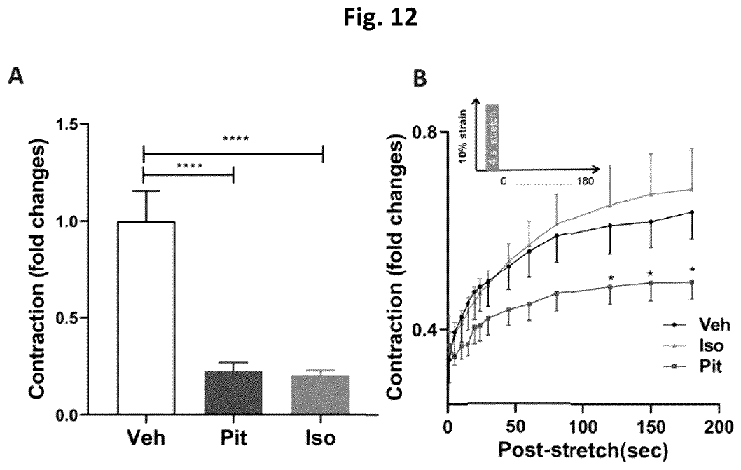

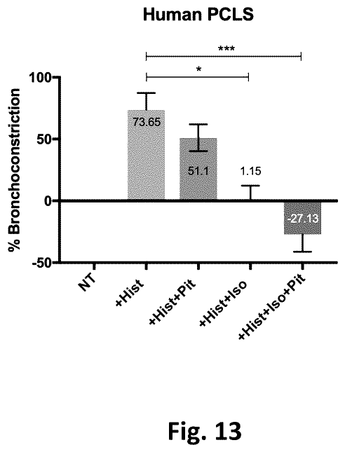

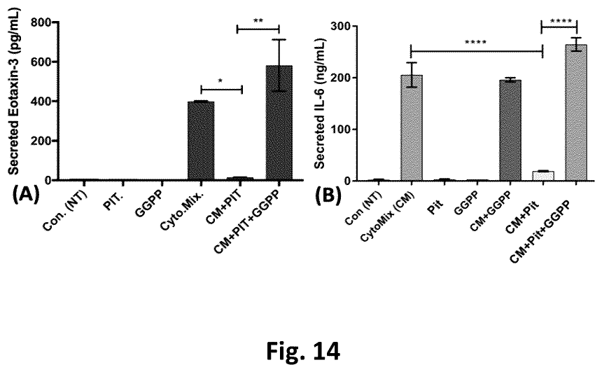

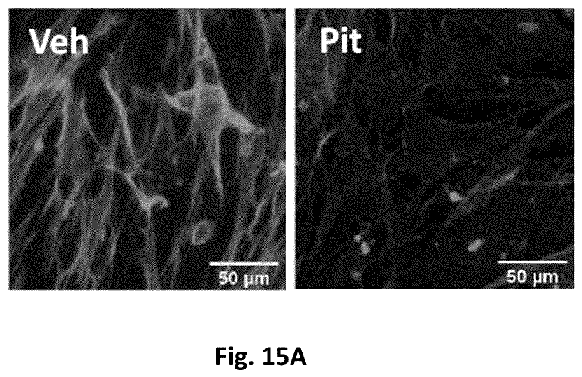

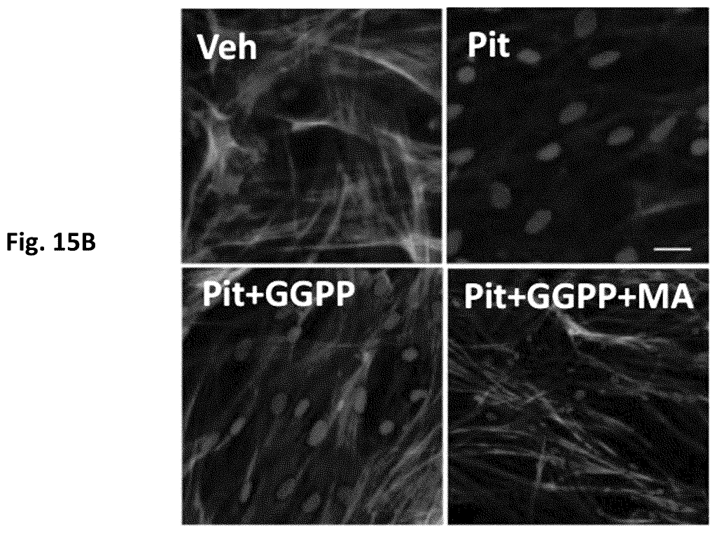

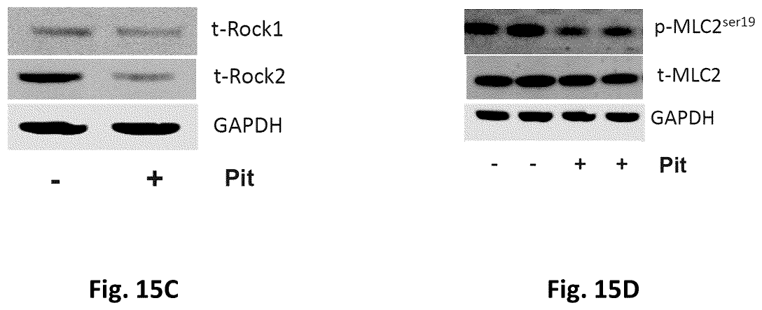

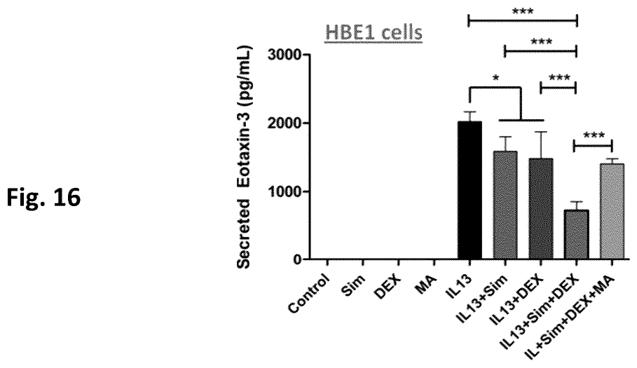

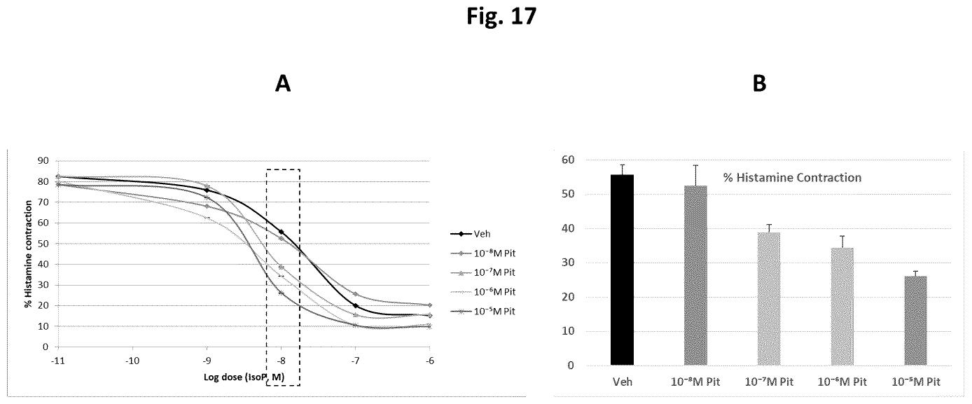

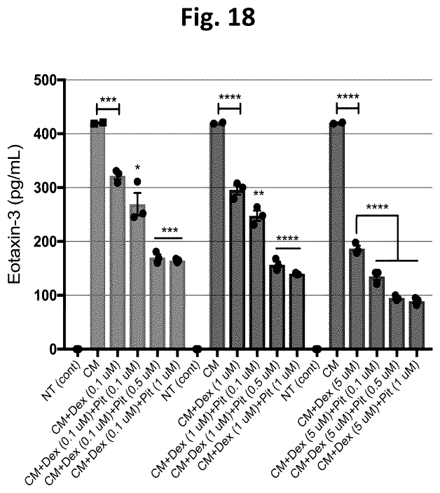

Ros: rosuvastatin; Sim: simvastatin (in the biologically active form, β-hydroxy acid, “SA”); Pit: pitavastatin. Pitavastatin and simvastatin were more potent at the concentrations used. FIG. 1A shows the effect in the absence of mevalonate. FIG. 1B shows the effect in the presence of 100 μM mevalonate, which abbrogates the beneficial effect of statins on ASM relaxation. This indicates that the statin effect occurs via inhibition of the mevalonate pathway. Of note, simvastatin is a prodrug (lactone form). Once absorbed, it is bio-transformed to the β-hydroxy simvastatin acid which is the active metabolite. In the blood circulation, there is a constant equilibrium between lactone and hydroxy acid.

FIG. 1C shows the statin dose-response across a range of drug lipophilicity. Simvastatin and pitavastatin are highly lipophilic, atorvastatin is of moderate lipophilicity, and pravastatin is the least lipophilic (most hydrophilic) statin. At a dose of 0.4 μM treated for 24 hours, simvastatin and pitavastatin display a significant reduction in strain energy (the energy imparted by the contracting cells upon the substrate=contraction) which equates to increased ASM relaxation, as compared to pravastatin which demonstrated no effect at these concentrations. At 2 and 10 μM, simvastatin, pitavastatin, and atorvastatin significantly (and further) reduce strain energy as compared to pravastatin. As compared to no treatment (0 μM), statistically significant reductions in strain energy occurred as follows: Simvastatin at 0.4 and 10 μM, pitavastatin at 2 and 10 μM, and atorvastatin at 2 and 10 μM. P-values: ***p<0.001, ****p<0.0001.

FIG. 1D shows that pitavastatin is a more potent inhibitor of ASM cell contraction than other statins. Pre-treatment (1 μM, 24 hours) with pitavastatin potently and significantly inhibits basal ASM contraction (a.k.a. strain energy), and pitavastatin was also more potent than simvastatin at the same dose, further confirming pitavastatin's enhanced potency as compared to the more lipophilic simvastatin. P-values: **p<0.01, ***p<0.001 compared to NT.

FIGS. 2A through 2E shows the results of testing for apoptosis and necrosis using statins as compared to a positive control which is known to induce apoptosis (FIG. 2A). In FIGS. 2B-E the individual statins are shown: (2B) simvastatin (SA), (2C) pitavastatin, (2D) rosuvastatin, and (2E) pravastatin. There was no evidence of apoptosis or cell death with the statin doses tested, including the doses where statins had a relaxing effect on ASM cells. Abbreviations: Sim (simvastatin), Pra (pravastatin), Pit (pitavastatin), Ros (rosuvastatin).

FIG. 3 shows the dose-dependent effects of simvastatin acid (SA) and pitavastatin on primary ASM cells obtained from three different human donors.

FIGS. 4A-4C show that statins inhibit histamine-induced ASM contraction. FIG. 4A: Pre-treatment (24 hrs) with pitavastatin potently and significantly inhibited both basal and histamine-induced (histamine at 10 μM for 30 min) ASM contraction, indicating that pitavastatin can prevent agonist-induced contraction and protect against airway narrowing. At a dose of 0.4 μM, pitavastatin renders histamine ineffective at inducing ASM contraction (basal vs. post-histamine, p=NS), with similar effects at higher doses (2, 100, 50 μM). A similar pattern was observed with simvastatin (FIG. 4B) and atorvastatin, with pitavastatin being most potent of the three drugs. Abbreviations: NT—no treatment, NS—not significant. P-values: *p<0.05, **p<0.01, ****p<0.0001, ####p<0.0001. FIG. 4C shows ASM relaxation over time. Pitavastatin caused greater ASM relaxation than simvastatin at 1 μM at all time points, including 24 hours, with or without media starvation. H=histamine applied. I=isoproterenol (β2 agonist) is applied. Histamine causes ASM contraction, while isoproterenol causes ASM relaxation. SE=strain energy (ASM contraction).

FIGS. 5A and 5B show that statins inhibit the contractile function in ASM cells. FIG. 5A shows that pitavastatin inhibits Rho kinase (ROCK-1) phosphorylation in a mevalonate-dependent manner in human ASM cells. Histamine (10 μM, 5 min.) induces ROCK-1 phosphorylation, and this is inhibited by pre-treatment (24 hrs) with pitavastatin (Pit, 1 μM). Co-treatment with mevalonate (MA, 200 μM, 24 hrs) abrogates the inhibitory effect of Pit on ROCK-1 phosphorylation. This confirmed that the MA pathway mediates ROCK-1 activation.

FIG. 5B shows that pitavastatin inhibits myosin light chain-2 (MLC-2) phosphorylation in human ASM cells. Thrombin (2 Units, 30 min) induced MCL-2 phosphorylation, and was inhibited by pre-treatment with pitavastatin (24 hrs, 1 or 10 μM). P-values: **p<0.01, ***p<0.001. The data shows that statins block the ASM contractile machinery. MLC proteins directly control smooth muscle contraction.

FIG. 6 shows that the bronchodilatory effects of intratracheally-instilled pitavastatin are independent of any anti-inflammatory effects. Using a non-inflammatory mouse model of methacholine (MCh)-induced hypercontractility, pre-treatment with intratracheal pitavastatin (5 mg/kg for 5 days) for 1 hr prior to each MCh nebulization caused a significant reduction in % contraction of airways (control 22.3% vs. statin 7.3%, *p=0.0361). Airway contraction was measured in response to 500 nM (0.5 μM) MCh using precision-cut lung slices (n=2 mice per group, 13 airways per group).

FIG. 7 shows the experimental design for the non-human primate inhaled statins trial.

FIG. 8 shows that inhaled (nebulized) simvastatin inhibits basal levels of eicosanoid lipids (LTB4 and TXB2) that cause broncoconstriction.

FIG. 9 shows the tissue distribution of inhaled simvastatin. Using mass spectrometry and metabolomics to study the tissue distribution of inhaled (nebulized) simvastatin (1 mg/kg) and its effects of lung lipids, simvastatin and its active metabolite simvastatin acid (SA) concentrate in the main stem bronchi and lower lung lobes (~0.8-1 μg/100,000 epithelial cells). Up to 405 ng/g SA was detected in the liver, while 25 ng/g and 7 ng/g SA were detected in the large intestine and muscle, respectively.

FIGS. 10A to 10F show that pitavastatin inhibits basal-, histamine-, and MCh-induced ASM contraction. FIG. 10A shows that, as compared to no treatment (0 μM), statistically significant reductions in contraction occurred as follows: Pitavastatin (Pit) at 0.4, 2 and 10 μM and Simvastatin (Sim) at 0.4 and 10 μM. Pravastatin had no beneficial effect on ASM cell relaxation. FIG. 10B shows that while both 1 μM Sim and 1 μM Pit reduced ASM contraction time-dependently compared to control, Pit was significantly more efficacious than Sim at 24 hrs (indicated by #). The experiment was performed under serum-deprived media conditions. FIG. 10C shows that 0.4 μM Pit inhibits histamine-induced ASM contraction while 0.4 μM Sim does not. Statistical comparison was performed using Student's t-test. FIG. 10D shows that the force inhibitory effects of 1 μM Pit was reversed when the wells were resuspended with media without Pit. FIG. 10E shows that statin treatment did not induce cellular apoptosis. Digitonin (50 μg/ml) was used as a positive control. FIG. 10F shows that, as compared to no treatment (0 μM), no reductions in viability were observed in mouse PCLS. 0.01% Triton® treatment for 2 hrs is included as a positive control. All cellular experiments were performed in serum (10% FBS)-containing media conditions, unless otherwise indicated. One non-asthmatic primary human ASM donor line was used in FIGS. 10A-D; FIG. 10E used one non-asthmatic hTERT ASM cell line. In all graphs, ASM contraction is plotted as fold change to the pre-treatment baseline value at 0 hr. p-values: *, #p<0.05; **, ##p<0.01; ###p<0.001. For each group, n=4-8 separate wells per condition. All data are reported as mean and standard error of the mean (SEM).

FIGS. 11A to 11C show that pitavastatin inhibits ASM contraction in human cells, human PCLS, and mice. FIG. 11A shows that ASM cells from asthmatics had greater basal contraction (in the absence of additional agonist) than non-asthmatic ASM cells. Despite these basal force differences, across both asthmatic and non-asthmatic donor cells, pitavastatin inhibited ASM contraction dose-dependently (p<0.0001 compared to 0 μM treatment). For each donor, n=4-8 separate wells per condition. FIG. 11B shows that in a non-inflammatory mouse model of MCh-induced ASM hypercontraction, pre-treatment with intratracheal pitavastatin (5 mg/kg for 5 days) for 1 hr prior to each MCh nebulization caused a significant reduction in % airway contraction (control=24.9%, vehicle=27.2%, pitavastatin=14.2%, *p<0.05). FIG. 11C shows that airways of human PCLS pre-treated with 5 μM Pit (n=5) or vehicle (n=3) for 24 hrs and post-treated with 0, 0.1, and 1 μM histamine (hist) for 15 min each exhibited reduced narrowing. As compared to vehicle, Pit significantly reduced 1 μM hist-induced bronchoconstriction. Changes in lumen narrowing are reported as % change in lumen area compared with 0 μM hist. The absolute value of lumen area was not statistically different between the Pit and vehicle groups at 0 μM histamine. All data are reported as mean and standard error of the mean (SEM).

FIGS. 12A to 12B show that pitavastatin potentiates the ASM relaxation effect of a simulated deep breath, a beneficial effect of pitavastatin that is notably absent for isoproterenol. FIG. 12A shows that, as compared to untreated controls (n=7), pre-treatment with 1 μM Pit (24 hrs) (n=7) or 10 μM isoproterenol (30 min) (n=6) significantly inhibited basal ASM contraction. Shown are contraction values normalized to the untreated control group. FIG. 12B shows that, in response to a subsequent single stretch-unstretch maneuver that mimics a deep breath (10% magnitude, 4-sec duration), the ASM cell promptly and dramatically ablated its contraction. The forces subsequently recovered over 180 seconds. While force ablation was similar across all three groups, the subsequent force recovery was significantly inhibited by pitavastatin treatment (*p<0.05; ****p<0.0001). n indicates the number of separate wells of ASM monolayers. All data are reported as mean and standard error of the mean (SEM).

FIG. 13 shows that both pitavastatin (Pit), isoproterenol (Iso), and the combination of Pit and Iso reduced histamine (Hist)-induced bronchoconstriction (*p=0.0298). This also shows that the combination is at least additive, and that pitavastatin does not interfere with the action of isoproterenol, and vice versa. Precision-cut human lung slices from one human donor lung were pre-treated with 5 μM Pitavastatin (Pita) or vehicle (control) for 24 hours and post-treated with histamine (hist, 10 μM for 15 min) followed by isoproterenol (iso, 30 μM for an additional 30 min). Changes in lumen narrowing are reported as percentage changes (±SEM) of the pre-treatment state. The absolute value of lumen airway area was not statistically different between the Pita and control groups at the pre-treatment state. The experiment was performed under serum-deprived media conditions. n=3-7 airways per group.

FIGS. 14A and 14B show that pitavastatin inhibits ASM cell secretion of pro-inflammatory cytokines in a GGPP-dependent manner. Non-asthmatic primary human ASM cells were grown to confluence and were either untreated (Con. (NT)) or pre-treated with 2 mM pitavastatin (Pit or PIT) and GGPP (10 mM) for a total of 72 hrs. Cells were treated with either IL17 and TNFα, or IL13 and TNFα, for 18 hrs at 10 ng/ml. FIG. 14A: PIT inhibited IL13/TNFα-induced eotaxin-3 peptide secretion by a GGPP-dependent mechanism. FIG. 14B: Pit inhibited IL17/TNFα-induced IL6 peptide secretion by a GGPP-dependent mechanism. The IL13/IL17/TNFα cocktail is denoted as “CytoMix (CM)” in the figures. All experiments were conducted under serum-containing media conditions (10% FBS). P-values: *p<0.05, **p<0.01, ****p<0.0001.

FIGS. 15A to 15C show that pitavastatin inhibits the ASM cytoskeleton via an MA- and GGPP-dependent mechanism. Non-asthmatic primary human ASM cells were treated either with 1 μM pitavastatin (Pit) in vehicle, or vehicle alone, for 24 hrs. Wells were then immunostained for F-actin expression. Pit significantly reduced basal F-actin expression (FIG. 15A). Non-asthmatic primary human ASM cells were co-treated with 1 μM pitavastatin (Pit), Pit with 10 μM GGPP, or Pit with 10 μM GGPP and 100 μM MA for 24 hrs. Pit reduced F-actin expression and ASM contraction: these reductions were abrogated by GGPP and MA (FIG. 15B). Cell lysates were analyzed by western blot for total ROCK-1 and total ROCK-2. Pit reduced the basal expression of both ROCK-1 and ROCK-2 (FIG. 15C). FIG. 15D shows that pitavastatin reduced the total expression of MLC-2 (total and phosphorylated).

FIG. 16 shows that simvastatin and dexamethasone synergistically inhibit eotaxin-3 secretion from HBE1 cells. Pre-treatment of HBE1 cells with simvastatin (Sim, 5 μM) and/or dexamethasone (DEX, 10⁻⁷ M) for 72 hours each independently inhibited IL13-induced eotaxin-3 extracellular secretion, simvastatin and dexamethasone together exhibited a synergistic inhibitory effect on eotaxin-3 secretion.

FIG. 17A shows that pre-treatment with appropriate concentrations of statin potentiates the relaxation effect of relevant concentrations of isoproterenol. FIG. 17B details the data shown in the boxed portion of FIG. 17A.

FIG. 18 shows that pre-treatment with appropriate concentrations of statin potentiates the relaxation effect of dexamethasone. Primary human airway smooth muscle cells were cultured to confluence in serum-containing media (10% FBS), then pre-treated with dexamethasone (“Dex”) at 0.1, 1, or 5 μM, with or without pitavastatin (“Pit”) at 0.1, 0.5, or 1 μM concentrations for 60 hrs. The ASM cells were then exposed to a mixture of cytokines (10 n/mL IL-13, IL-17, and TNFα, “CM”) for 15 hrs, and the expression of eotaxin-3 was measured. “NT” means no treatment. Significant reductions in eotaxin-3 expression were observed when pitavastatin was added to any concentration of dexamethasone.

### DETAILED DESCRIPTION

### General

The need for novel bronchodilators is met by new methods using statins, which offer a new mechanism to bronchodilate airways to improve lung function and reduce symptoms when delivered directly to the airways by inhalation. The present disclosure shows that inhaled statins have therapeutic effects separate and apart from any anti-inflammatory activity. Administration of statins directly to the airways delivers an effective amount of the statin to the airway smooth muscle (ASM), which is not attained using oral administration. When inhaled, however, statins are able to induce ASM relaxation, reduce bronchoconstriction and airway mucus accumulation, reduce bronchospasm, reduce hyperresponsiveness and hypercontraction, potentiate deep breath-induced ASM relaxation and bronchodilation, potentiate the bronchodilatory effects of agents such as β2-agonists and inhaled corticosteroids, reduce the need to use inhaled corticosteroids, maintain pulmonary function, and improve pulmonary function. Accordingly, the present disclosure includes new methods and formulations for treating lung airway diseases and disorders. Inhaled statins directly reduce the contractile force exerted by ASM, inhibit the ASM cytoskeleton, and inhibit the release of inflammatory cytokines and mediators by ASM. Inhaled statins, in combination with additional therapeutic agents, can increase the therapeutic effect of the additional therapeutic agents, which permits dose reduction and/or increased efficacy.

### Definitions

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

“Statins” are small molecule HMG-COA reductase inhibitors. Statins were designed to block the mevalonate metabolic pathway, and thereby reduce the production of cholesterol in the body. Suitable statins of the disclosure include, without limitation, simvastatin, pitavastatin, rosuvastatin, atorvastatin, lovastatin, fluvastatin, mevastatin, cerivastatin, tenivastatin, and pravastatin, and isomers, enantiomers, and diastereomers thereof. Hydrophobic statins include simvastatin, pitavastatin, and other statins with a similar hydrophobicity. Hydrophilic statins include pravastatin, and other statins with a similar hydrophilicity.

The term “airway smooth muscle” (“ASM”) refers to the smooth, involuntary muscle tissue that lines the bronchi and bronchioles. ASM contraction reduces the diameter of airways, while ASM relaxation dilates the airways.

The term “therapeutically effective amount” refers to the amount of statin (or an isomer, enantiomer, diastereomer) or mixture thereof that is sufficient to achieve a measurable beneficial effect when administered by inhalation. The beneficial effect may be reduction of airway smooth muscle contraction, the reduction of bronchospasm or bronchoconstriction, the prophylactic maintenance of lung function, the reduction of mucus accumulation, the reduction of corticosteroids required to control symptoms, the reduction in severity and/or frequency of asthma exacerbations, improvement in breathing-induced bronchodilation, and the like.

A “sub-therapeutic dose” refers to the dose of one or more agents in a synergistic or potentiated combination formulation, method, or system, wherein the dose of the agent is reduced to a level that would be insufficient or sub-therapeutic when administered alone or as part of a non-synergistic combination formulation, method, or system, but is sufficient for therapeutic use when administered as part of the synergistic combination formulation, method, or system. The sub-therapeutic dose of an agent can be about 90%, 80%, 75%, 70%, 65%, 60%, 55%, 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% of the effective dose of the agent when administered by inhalation as part of a non-synergistic formulation, method, or system according to the present disclosure.

The term “pharmaceutically acceptable carrier” refers to an excipient that is non-toxic to the subject at the amount and concentration in which it is administered, within which the statin may be dissolved and/or suspended. In the practice of the instant disclosure, pharmaceutically acceptable carriers are suitable for administration by inhalation.

The term “lung airway disease” refers to a disease or disorder in which obstruction, or restriction or interference with airflow into and out of the lung, is a substantial symptom. This obstruction may result from constriction of ASM (bronchoconstriction) and/or over secretion of mucus. Lung airway diseases include, without limitation, asthma; exercise-induced bronchoconstriction (or exercise-induced asthma); chronic obstructive pulmonary disease (COPD) which may include emphysema, chronic bronchitis, and/or alpha-1 antitrypsin deficiency (AATD); asthma-COPD overlap syndrome (ACOS) (also known as asthma-COPD overlap or ACO); cystic fibrosis; acute bronchitis; eosinophilic bronchitis; constrictive bronchiolitis; and bronchiectasis. The use of inhaled statins can reduce compressive forces, and thereby reduce airway remodeling, mucus hypersecretion, and mucus plug formation. Lung airway diseases that are considered “non-inflammatory” include exercise-induced bronchospasm, exercise-induced asthma, aspirin-exacerbated respiratory disease, NSAID-exacerbated respiratory disease, paucigranulocytic asthma, obesity-associated airway hyperresponsiveness, post-viral airway hyperresponsiveness, and other lung airway diseases that are not initiated or maintained by inflammation.

“Bronchoprotection” is a lung “protective” activity or administration using a method, formulation, or system of the disclosure to reduce the future symptoms or harm from an effect that has already occurred or is expected to be experienced in the future, where the subject receiving the method, formulation, or system is at risk of experiencing the future symptoms. These future symptoms can include post-infectious bronchospasm due to viral, bacterial, fungal, and/or mycobacterial infection; airway edema due to congestive heart failure; airway edema due to pulmonary edema; airway edema due to cardiogenic pulmonary edema; airway edema due to non-cardiogenic pulmonary edema; bronchiolitis due to airway edema; bronchiectasis due to anatomic distortions rather than inflammation; foreign body aspiration; aspiration of food, liquids, and/or gastric contents; gastro-esophageal reflux disease; lung cancer or metastatic cancer to the lung causing local edema and bronchospasm; pulmonary embolism (which can release local factors that cause wheezing due to bronchospasm); airway trauma, including surgery; anaphylaxis and anaphylactoid reactions; neurally mediated cough and/or bronchospasm; inhalation injury-associated bronchospasm; endocrine dysfunction associated bronchospasm; paraneoplastic syndrome-associated bronchospasm; and other events that are likely to cause bronchospasm. A subject is “expected to” experience future symptoms if the subject is at least 10%, 20%, 30%, 40%, 50%, 60%, 70%, 80%, 90%, or about 100% likely to experience one or more of the symptoms, due to the subjects, health, physical condition, genetics, occupation, age, or any other factor causally related to the expected future symptoms.

A lung airway disease is “characterized” by a given factor if that factor is characteristic of the disease, i.e., if the disease is usually associated with that factor, regardless of whether or not the factor is also found in other diseases. For example, asthma may be characterized by bronchoconstriction because bronchoconstriction appears in a majority of asthma cases, regardless of the fact that bronchoconstriction is also characteristic of emphysema.

An “interstitial lung disease” is one occurs in the lung tissue and spaces between the lung airways, for example, the basement membrane, perivascular, and perilymphatic tissues. Despite the fact that these diseases do not directly affect lung airways, they may still have indirect effects and symptoms that impact the airways and pulmonary function.

### Formulations

The disclosed compositions are formulated to be suitable for inhalation, in which the composition is inhaled or sprayed into the lungs. Ideally, the composition is administered in such a manner that it is distributed evenly throughout the airways, providing an effective amount of statin directly to the ASM. This is generally accomplished by administering the formulation as a population of small particles suspended in air or a gas, where the distribution of particle sizes affects the distance that the particles will penetrate distal to the trachea. The composition may be in the form of a solution, suspension, powder, or other suitable form for pulmonary administration. See, for example, H. M. Mansour et al., Int J Nanomed (2009) 4:299-319. These compositions are administered to the lungs, for example, in an aerosol, atomized, nebulized, or vaporized form through appropriate devices known in the art. The amount of the composition administered can be controlled by providing a valve to deliver a metered amount, as in a metered dose inhaler (MDI) that delivers a fixed dose in a spray with each actuation of the device. In this way, an appropriate dose (e.g., a therapeutically effective amount) of the composition can be delivered reliably from a device that contains multiple doses.

The formulation employed for delivery will typically be designed to work with a particular mode of administration, such as an aerosol formulation, a nebulizer formulation, or a dry powder formulation.

Formulations of the disclosure contain a therapeutically effective amount of a statin. In some embodiments, the therapeutically effective amount is at least about 0.005, 0.01, 0.02, 0.03, 0.04, 0.05, 0.06, 0.07, 0.08, 0.09, 0.1, 0.2, 0.3, 0.4, 0.5, 0.6, 0.7, 0.75, 0.8, 0.9, 1.0, 1.5, 2.0, 3.0, 4.0, 5.0, 6.0, 7.0, 8.0, 9.0, 10, 12, 14, 15, 17, 20, 25, 30, 35, 40, 45, 50, 55, 60, 65, 70, 75, 80, 85, 90, 95, or 100 μg. In some embodiments, the therapeutically effective amount is at least about 0.1, 0.2, 0.3, 0.4, 0.5, 0.6, 0.7, 0.75, 0.8, 0.9, 1.0, 1.5, 2.0, 3.0, 4.0, 5.0, 6.0, 7.0, 8.0, 9.0, 10, 12, 14, 15, 17, 20, 25, 30, 35, 40, 45, 50, 55, 60, 65, 70, 75, 80, 85, 90, 95, or 100 mg. In some embodiments, the therapeutically effective amount will be no greater than about 100, 95, 90, 85, 80, 75, 70, 65, 60, 55, 50, 45, 40, 35, 30, 25, 20, 15, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, or 0.005 mg.

The formulation can contain any pharmaceutically active statin or a mixture thereof. In some embodiments, the statin is selected from the group consisting of simvastatin, pitavastatin, rosuvastatin, atorvastatin, lovastatin, fluvastatin, mevastatin, cerivastatin, tenivastatin, and pravastatin, and isomers, enantiomers, and diastereomers thereof. In some embodiments, the statin is selected from the group consisting of simvastatin, pitavastatin, atorvastatin, lovastatin, and pravastatin. In some embodiments, the statin is selected from the group consisting of simvastatin and pitavastatin.

Formulations of the disclosure may further include an additional therapeutic agent, which can be selected from β-agonists; corticosteroids; muscarinic antagonists; RhoA inhibitors; GGTase-I or -II inhibitors; ROCK1 and/or ROCK2 inhibitors; soluble epoxide hydrolase inhibitors; fatty acid amide hydrolase inhibitors; leukotriene receptor antagonists; phosphodiesterase-4 inhibitors such as roflumilast; 5-lipoxygenase inhibitors such as zileuton; mast cell stabilizers such as nedocromil; theophylline; anti-IL5 antibodies or antibody derivatives; anti-IgE antibodies or antibody derivatives; anti-IL5 receptor antibodies or antibody derivatives; anti-IL13/4 receptor antibodies or antibody derivatives; biologics such as mepolizumab, reslizumab, benralizumab, omalizumab, and dupilumab; β-agonist and muscarinic antagonist combinations, including both long- and short-acting formulations; β-agonist and corticosteroid combinations, including both long- and short-acting formulations; corticosteroids and muscarinic antagonist combinations, including both long- and short-acting formulations; and β-agonist, corticosteroid, and muscarinic antagonist combinations, including both long- and short-acting formulation.

An antibody derivative is a protein capable of binding an antigen that is similar to or based on an antibody. Examples of antibody derivatives include nanobodies, diabodies, triabodies, minibodies, F(ab′)2 fragments, F(ab)v fragments, single chain variable fragments (scFv), single domain antibodies (sdAb), and functional fragments thereof.

As the additional therapeutic agent is also not subject to hepatic first pass metabolism, it too may be administered at doses that are generally lower than the dose effective in oral or parenteral administration. In some embodiments, the effective dose when administered by inhalation is less than about 90%, 80%, 75%, 70%, 65%, 60%, 55%, 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% of the dose normally recommended for oral administration.

Suitable corticosteroids for use as an additional therapeutic agent include, without limitation: beclomethasone, fluticasone, budesonide, mometasone, flunisolide, alclometasone, beclometasone, betamethasone, clobetasol, clobetasone, clocortolone, desoximetasone, dexamethasone, diflorasone, difluocortolone, flurclorolone, flumetasone, fluocortin, fluocortolone, fluprednidene, fluticasone, fluticasone furoate, halometasone, meprednisone, mometasone, mometasone furoate, paramethasone, prednylidene, rimexolone, ulobetasol, amcinonide, ciclesonide, deflazacort, desonide, formocortal, fluclorolone acetonide, fludroxycortide, fluocinolone acetonide, fluocinonide, halcinonide, and triamcinolone acetonide.

Muscarinic antagonists are anticholinergic agents that block the muscarinic acetylcholine receptor, and can therefor block bronchoconstriction. Suitable muscarinic antagonists for use as an additional therapeutic agent include, without limitation: ipratropium bromide, tiotropium, glycopyrrolate, glycopyrronium bromide, revefenacin, umeclidinium bromide, aclidinium, trospium chloride, oxitropium bromide, oxybutynin, tolterodine, solifenacin, fesoterodine, and darifenacin.

Beta-agonists are compounds that activate β2-adrenergic receptors, and are used to relax ASM. Suitable beta-agonists (β-agonists) for use as an additional therapeutic agent include, without limitation: albuterol, arformoterol, buphenine, clenbuterol, bopexamine, epinephrine, fenoterol, formoterol, isoetarine, isoproterenol, orciprenaline, levoalbutamol, levalbuterol, pirbuterol, procaterol, ritodrine, albuterol, salmeterol, terbutaline, arbutamine, befunolol, bromoacetylalprenololmenthane, broxaterol, cimaterol, cirazoline, etilefrine, hexoprenaline, higenamine, isoxsuprine, mabuterol, methoxyphenamine, oxyfedrine, ractopamine, reproterol, rimiterol, tretoquinol, tulobuterol, zilpaterol, and zintero.

ROCK inhibitors inhibit the enzyme Rho Kinase (ROCK1 and/or ROCK2). Suitable ROCK inhibitors include, for example, 1-methyl-5-(1H-pyrrolo[2,3-b]pyridin-4-yl)-1H-indazole (“TS-f 22”, M. Shen et al., Sci Rep (2015) 5:16749), (1S)-2-amino-1-(4-chlorophenyl)-1-[4-(1H-pyrazol-4-yl)phenyl]ethanol (“AT13148”, T. A. Yap et al., Clin Cancer Res (2012) 18(14):3912-23), N-(6-fluoro-1H-indazol-5-y1)-6-methyl-2-oxo-4-[4-(trifluoromethyl)phenyl]-3,4-dihydro-1H-pyridine-5-carboxamide (“GSK429286A”, E. Ahler et al., Mol Cell (2019) 74(2):393-408e20), 1-[(3-hydroxyphenyl)methyl]-3-(4-pyridin-4-yl-1,3-thiazol-2-yl)urea (“RKI-1447,” H. Wang et al., Cancer Res (2017) 77(8):2148-60), and 4-[(1R)-1-aminoethyl]-N-pyridin-4-ylcyclohexane-1-carboxamide (“Y-27632”, Y-C. Liao et al., Cell (2019) 179(1):147-64.e20). Suitable RhoA inhibitors include compounds such as N-[1-(4-chloroanilino)-1-oxopropan-2-yl]oxy-3,5-bis(trifluoromethyl)benzamide (“CCG-1423”, D. A. Lionarons et al., Cancer Cell (2019) 36(1):68-83.e9). Suitable GGTI inhibitors include compounds such as N-(1-amino-1-oxo-3-phenylpropan-2-y1)-4-[2-(3,4-dichlorophenyl)-4-(2-methylsulfanylethyl)-5-pyridin-3-yl-pyrazol-3-yl]oxybutanamide (“GGTI-DU40”, Y. K. Peterson et al., J Biol Chem (2006) 281:12445-50), and (2S)-2-[[4-[[(2R)-2-amino-3-sulfanylpropyl]amino]-2-naphthalen-1-yl-benzoyl]amino]-4-methylpentanoic acid 2,2,2-trifluoroacetic acid (“GGTI-297”, P. A. Subramani et al., Bioinformation (2015) 11(5):248-53). Suitable soluble epoxide hydrolase inhibitors include compounds such as, for example, 1-(1-acetylpiperidin-4-yl)-3-(1-adamantyl)urea (“AR9281”, R. H. Ingraham et al., Curr Med Chem (2011) 18(4):587-603), 1-(1-propanoyl-piperidin-4-yl)-3-[4-(trifluoromethoxy)phenyl]urea (“TPPU”, Y-M. Kuo et al., Mol Neurobiol (2019) 56:8451-74).

Suitable fatty acid amide hydrolase inhibitors include, without limitation, compounds such as 4-hydroxy-N-[(5Z,8Z,11Z,14Z)-icosa-5,8,11,14-tetraenyl]benzamide (“AM-1172”, C. J. Hillard et al., J Mol Neurosci (2007) 33:18-24), N-Phenyl-4-(3-phenyl-1,2,4-thiadiazol-5-y1)-1-piperazinecarboxamide (“JNJ 1661010”, T. Lowin et al., Arth Res Ther (2015) 17:321), and N-3-pyridinyl-4-[[3-[[5-(trifluoromethyl)-2-pyridinyl]oxy]phenyl]methyl]-1-piperidinecarboxamide (“PF-3845”, S. Ghosh et al., J Pharmacol Exp (2015) 354(2):111-20). Suitable leukotriene receptor antagonists include, without limitation, compounds such as zafirlukast, montelukast, and zileuton.

### (a) Aerosol Formulations

Aerosols are suspensions of small solid particles or liquid droplets, typically having an average diameter <10 um, suspended in air or another gas. Aerosol formulations for delivering drugs to the respiratory tract are known in the art. See for example, A. Adjei et al., J Pharm Res (1990) 1:565-69; P. Zanen et al., J Int J Pharm (1995) 114:111-15; I. Gonda, Crit Rev Ther Drug Carrier Syst (1990) 6:273-313; Anderson et al., Am Rev Respir Dis, (1989)140:1317-24; the contents of all of which are herein incorporated by reference in their entirety.

Compositions for aerosol administration via pressurized metered dose inhalers (pMDIs) can be formulated as solutions or suspensions. Solution compositions can be more convenient to manufacture, as the active agent is completely dissolved in the propellant vehicle and avoids the physical stability problems (such as particle aggregation) sometimes associated with suspension compositions. If the agent is not sufficiently soluble in the propellant, a co-solvent such as ethanol can be used to provide enhanced solubility in a pharmaceutical composition for administration by pMDI. In some embodiments, the formulation comprises a statin dissolved in a propellant and a co-solvent.

Suspension formulations can include small, solid particles of the pharmaceutical agent, typically having an average diameter of less than about 10 μm. Such formulations can be prepared by grinding or milling a crystalline form of the agent, or by spray-drying a solution containing the agent. In some embodiments, the formulation comprises a powdered statin, a propellant, and a suspending vehicle. In some embodiments, the suspending vehicle is selected from PEG400, PEG1000, and propylene glycol (1,2-propane diol). In some embodiments, the statin comprises pitavastatin.

The pharmaceutical compositions may be formulated with one or more suitable propellants, such as, for example, hydrofluoroalkanes, CO₂, or other suitable gases. In some embodiments, a surfactant may be added to reduce the surface and interfacial tension between the composition, the propellant, and the co-solvent, if present. The surfactant may be any suitable, non-toxic compound which is non-reactive with the other pharmaceutical composition components and which reduces the surface tension and/or interfacial tension between the composition, the propellant, and co-solvent to the desired degree. In some embodiments, the formulations do not require a surfactant to produce and/or maintain a stable pharmaceutical composition solution under normal operating conditions, and may be surfactant-free.

### (b) Nebulizer Formulations

“Nebulization” refers to reduction of a liquid to a fine spray or mist. Small liquid droplets of uniform size are produced from a larger body of a liquid formulation in a controlled manner, typically having an average particle size of about 0.5 μm to about 10 μm. Nebulization can be achieved by any suitable means, including a mechanical nebulizer, such as a Respimat® Soft Mist nebulizer in which the formulation is squeezed through nozzles under spring pressure; a jet nebulizer, in which a compressor compresses air or oxygen to flow through the liquid at high velocity, forming a mist; an ultrasonic wave nebulizer, in which a piezoelectric transducer oscillating at an ultrasonic frequency is placed in contact with the liquid formulation, the vibration forming a mist or aerosol; or a vibrating mesh nebulizer, in which a mesh or membrane with small holes is vibrated at the surface of the liquid reservoir, forming a fine mist. Nebulizers using any of these techniques are commercially available. When the active ingredients are adapted to be administered, either together or individually, via nebulizer(s) they can be in the form of a nebulized aqueous suspension or solution, with or without a suitable pH or tonicity adjustment, either as a unit dose or multidose device.

Formulations used in nebulizer administration are typically, but not necessarily, mainly aqueous solutions. In cases in which the agent to be administered is only sparingly soluble in water, pharmaceutically acceptable co-solvents such as ethanol can be added to dissolve or help dissolve the agent. Alternatively, the formulation can be a suspension of suitably sized particles suspended in a mainly aqueous carrier. Agents can also be formulated as solid lipid microparticles (SLM), solid lipid nanoparticles (SLN), or liposomes, and suspended in a liquid carrier for nebulization or aerosolization. See, e.g., M. Paranjpe et al., Int J Mol Sci (2014) 15:5852-73; M. J. de Jesús Valle et al., J Antibiot (Tokyo) (2013) 66(8):447-51, both incorporated herein by reference. The particle size of the nebulized droplets can be adjusted by a number of parameters, including for example the formulation viscosity and surface tension, and the nebulizer characteristics, as is taught in the art.

### (c) Dry Powder Formulations

Dry powder formulations, as the name implies, do not have a liquid carrier. Instead, the active agent and excipients are ground or milled to a fine powder, having a particle size suitable for inhalation. The formulation is designed to be carried into the lungs by a sharp inhalation and/or a puff of compressed air or gas. Dry powder formulations are particularly convenient when administering agents that are difficult to dissolve or suspend in conventional liquid carriers.

Dry powder formulations often contain excipients in addition to the active agent or agents. These excipients are often included to improve the flow properties of the product, including the dispersion and absorption, as well as for chemical stability during storage. The formulations can be prepared, for example, by spray-drying (A. A. Ambike et al., Pharm Res (2005) 22(6):990-98), grinding or milling, extrusion, precipitation, and/or screening using methods known in the art to obtain an inhalable powder. The excipients used may also be mixtures of ground excipients which are obtained by mixing excipient fractions of different mean particle sizes.

Examples of physiologically acceptable excipients which may be used to prepare the inhalable powders for use in the inhalers (or cartridges thereof) include monosaccharides (e.g., glucose, fructose or arabinose), disaccharides (e.g., lactose, saccharose, maltose, trehalose), oligo- and polysaccharides (e.g., dextrans, dextrins, maltodextrin, starch, cellulose), polyalcohols (e.g., sorbitol, mannitol, xylitol), cyclodextrins (e.g., α-cyclodextrin, β-cyclodextrin, χ-cyclodextrin, methyl-β-cyclodextrin, hydroxypropyl-β-cyclodextrin, sulfobutyl-β-cyclodextrin (Captisol®, Dexolve®)), amino acids (e.g., arginine hydrochloride), and salts (e.g., sodium chloride, calcium carbonate), or mixtures thereof. Lactose, glucose, and other compounds can be used in the form of their hydrates. The excipients can be combined with the statin before, during, or after the powdering process.

Within the scope of the inhalable powders, the excipients can have a maximum average particle size of up to about 250 μm, between 10 and 150 μm, or between 15 and 80 μm. Finer excipient fractions with an average particle size of 1 to 9 μm can also be added to the excipients mentioned above. The average particle size may be determined using methods known in the art (for example WO 02/30389). Finally, in order to prepare inhalable powders, a micronised crystalline statin, which can be characterized by an average particle size of about 0.5 to about 10 μm, or from about 1 to about 5 μm, is added to the excipient mixture (see, for example, WO 02/30389). Processes for grinding and micronizing active substances are known in the art. If no specifically prepared excipient mixture is used as the excipient, excipients which have a mean particle size of 10-50 μm and a 10% fine content of 0.5 to 6 μm can be used. In some embodiments, the maximum average particle size is less than about 250, 225, 200, 190, 180, 170, 160, 150, 140, 130, 125, 120, 115, 110, 105, 100, 95, 90, 85, 80, 75, 70, 65, 60, 55, 50, 45, 40, 35, 30, 25, 20, 19, 18, 17, 16, 15, 14, 13, 12, 11, 10, 9, 8, 7, 6, 5, 4, 3, 2, or 1 μm. In some embodiments, the average particle size is at least about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 12, 15, 17, 19, 20, 25, 30, 35, 40, 45, or 50 μm. In some embodiments, the average particle size is less than about 250, 225, 200, 190, 180, 170, 160, 150, 140, 130, 125, 120, 115, 110, 105, 100, 95, 90, 85, 80, 75, 70, 65, 60, 55, 50, 45, 40, 35, 30, 25, 20, 19, 18, 17, 16, 15, 14, 13, 12, 11, 10, 9, 8, 7, 6, 5, 4, 3, 2, or 1 μm.

In one method for preparing a dry powder formulation, the excipient and the active agent are placed in a suitable mixing container. In some embodiments, the active agent has an average particle size of 0.5 to 10 μm, 1 to 6 μm, or 2 to 5 μm. The excipient and the active agent are added using a sieve or a granulating sieve with a mesh size of 0.1 to 2 mm, 0.3 to 1 mm, or 0.3 to 0.6 mm. The excipient may be added first, and then the active agent is added to the mixing container. During this mixing process the two components may be added in batches, and the two components sieved in alternate layers. The mixing of the excipient with the active agent may take place while the two components are still being added.

Inhalable powders can also be formulated as PulmoSpheres (see, e.g., J. G. Weers et al., Ther Deliv (2014) 5(3):277-95; J. G. Weers et al., AAPS PharSciTech (2019) 20(3):103; and U.S. Pat. No. 9,452,139, all incorporated herein by reference), in which suspensions of micronized drug particles are spray-dried to form a powder. Alternatively, powders and suspensions can be formulated from self-assembling nanoparticles (see for example, N. J. Kenyon et al., PLOS One (2013) https://doi.org/10.1371/journal.pone.0077730).

### Inhalers

The three primary types of inhaler are the nebulizer, the pressurized metered-dose inhaler (pMDI), and the dry powder inhaler (DPI). Nebulizers convert a liquid solution or suspension of drug into a fine mist of droplets, which are then inhaled into the lungs. Nebulizers typically take longer to administer a drug than pMDIs or DPIs, and are less accurate in terms of the exact dose of drug that is absorbed, due to losses of drug in the device and to the surrounding air. However, they are typically the most easy to use, and can be used with subjects who are too young to operate pMDIs or DPIs, or who are unconscious. Nebulizers typically comprise a reservoir that contains the drug formulation, a nebulization chamber, a face mask, and a mechanism for nebulizing the formulation. In jet nebulizers, the mechanism comprises a nozzle through which air is passed at high velocity, which draws the liquid formulation up through a capillary tube. Droplets of the formulation are entrained in the air jet, and impacted against baffles which reduce the droplet size and/or screen out overly large droplets. The baffles also reduce the air speed, so that the resulting mist leaves the nebulizer at lower velocity and is more likely to reach the lower airways. The process of nebulization in these devices also usually reduces the temperature of the formulation, due to the evaporation of the droplets. Jet nebulizers typically require a compressor to generate the air flow, which makes them noisier and less portable than other inhalers.

Ultrasonic nebulizers employ an element that is vibrated at ultrasonic frequencies to break the liquid formulation into droplets. The vibrating element is often a stiff mesh or perforated membrane. These nebulizers are generally quieter than jet nebulizers, and do not require a compressor, although they do still require a power source. The ultrasonic vibration often raises the temperature of the formulation.

pMDIs contain a solution or suspension of drug in a propellant under pressure, and comprise a valve that delivers a precisely measured amount of the formulation when actuated. The propellant is often a gas such as a hydrofluoroalkane propellant, which is combined with the drug and optionally a co-solvent such as ethanol and/or a surfactant. The formulation is compressed into a liquid state, and loaded into the pMDI or a pMDI cartridge. A typical pMDI releases the formulation in liquid form into a metering chamber, which determines the amount of the dose. When the device is actuated, the measured formulation is released into an expansion chamber where the propellant is volatilized. For efficient and consistent delivery of the drug, the subject using the pMDI must coordinate his or her breathing with the device actuation, to insure that the greatest amount of aerosol possible reaches the lower airways. Modern pMDIs may further include valves or sensing mechanisms that release the aerosol only when the subject is inhaling. Most pMDIs also employ a spacer, which is essentially a tube between the pMDI and the subject, which improves the efficiency of aerosol delivery and permits more time for the propellant to evaporate (leading to smaller droplets).

DPIs, in general, contain a measured quantity of the drug as a dry powder, optionally having a dry powder carrier such as powdered lactose. DPIs rely on a sharp inhalation by the subject to dispense the powdered formulation, rather than forming a mist or aerosol. They are in general easier to use than pMDIs, although the efficiency of delivery depends in part on the airspeed that the subject is capable of producing. Newer DPIs that are breath-triggered but power assisted are in development.

Formulations of the disclosure can be administered using commercially available inhalation devices, such as nebulizers, for example without limitation, a Respimat® Soft Mist™ inhaler; inhalers such as a RespiClick® inhaler, Breezhaler® inhaler, Genuair® inhaler, an Ellipta® inhaler, and the like. Inhalers can be provided pre-filled, containing one or multiple therapeutic doses of a formulation of the disclosure, or can be configured to accept a cartridge that is pre-filled with one or multiple therapeutic doses of a formulation of the disclosure.

Inhalable powders and aerosols may, for example, be administered using inhalers which meter a single dose from a reservoir by means of a measuring chamber (see, e.g., U.S. Pat. No. 4,570,630) or by other means (see, e.g., DE 3625685). In some embodiments, the inhalable powders are packed into capsules or cartridges, which are used in inhalers such as those described in WO 94/28958.

Capsules and cartridges for use in an inhaler may be formulated containing a powder mix of the disclosed compounds or pharmaceutical compositions and a suitable powder base such as lactose or starch.

### Systems

Methods of the disclosure can also be practiced using systems of the disclosure, comprising a statin or a statin formulation, and one or more additional therapeutic agents or formulations comprising one or more additional therapeutic agents. In a system of the disclosure, the statin and the additional therapeutic agent(s) need not be present in the same formulation, and can be administered at different times. In some embodiments, the system comprises a statin selected from the group consisting of simvastatin, pitavastatin, rosuvastatin, atorvastatin, lovastatin, fluvastatin, mevastatin, cerivastatin, tenivastatin, and pravastatin, and isomers, enantiomers, and diastereomers thereof. In some embodiments, the statin is selected from the group consisting of simvastatin, pitavastatin, lovastatin, fluvastatin, mevastatin, cerivastatin, and tenivastatin. In some embodiments, the statin is a hydrophobic statin. In some embodiments, the statin is simvastatin or pitavastatin. In some embodiments, the statin is pitavastatin. In some embodiments, the statin is simvastatin.

In some embodiments, the formulation is a dry powder formulation. In some embodiments, the formulation is an aerosol formulation. In some embodiments, the formulation is a nebulizable formulation. In some embodiments, the nebulizable formulation comprises an aqueous solution of the statin. In some embodiments, the nebulizable formulation further comprises a pharmaceutically acceptable alcohol. In some embodiments, the pharmaceutically acceptable alcohol comprises ethanol.

The additional therapeutic agent may be any of the additional therapeutic agents described in the disclosure. In some embodiments, the additional therapeutic agent is beclomethasone, fluticasone, budesonide, mometasone, flunisolide, alclometasone, beclometasone, betamethasone, clobetasol, clobetasone, clocortolone, desoximetasone, dexamethasone, diflorasone, difluocortolone, flurclorolone, flumetasone, fluocortin, fluocortolone, fluprednidene, fluticasone, fluticasone furoate, halometasone, meprednisone, mometasone, mometasone furoate, paramethasone, prednylidene, rimexolone, ulobetasol, amcinonide, ciclesonide, deflazacort, desonide, formocortal, fluclorolone acetonide, fludroxycortide, fluocinolone acetonide, fluocinonide, halcinonide, or triamcinolone acetonide, or a combination thereof. In some embodiments, the additional therapeutic agent is albuterol, arformoterol, buphenine, clenbuterol, bopexamine, epinephrine, fenoterol, formoterol, isoetarine, isoproterenol, orciprenaline, levoalbutamol, levalbuterol, pirbuterol, procaterol, ritodrine, albuterol, salmeterol, terbutaline, arbutamine, befunolol, bromoacetylalprenololmenthane, broxaterol, cimaterol, cirazoline, etilefrine, hexoprenaline, higenamine, isoxsuprine, mabuterol, methoxyphenamine, oxyfedrine, ractopamine, reproterol, rimiterol, tretoquinol, tulobuterol, zilpaterol, or zintero, or a combination thereof. In some embodiments, the additional therapeutic agent is albuterol.

In some embodiments, the additional therapeutic agent is ipratropium bromide, tiotropium, glycopyrrolate, glycopyrronium bromide, revefenacin, umeclidinium bromide, aclidinium, trospium chloride, oxitropium bromide, oxybutynin, tolterodine, solifenacin, fesoterodine, darifenacin, or a combination thereof. In some embodiments, the additional therapeutic agent is roflumilast, zileuton, nedocromil, theophylline, an anti-IL5 antibody or antibody derivative, an anti-IgE antibody or antibody derivative, an anti-IL5 receptor antibody or antibody derivative, an anti-IL13/4 receptor antibody or antibody derivative, mepolizumab, reslizumab, benralizumab, omalizumab, dupilumab, or a combination thereof. In some embodiments, the additional therapeutic agent is TS-f22, AT13148, GSK429286A, RKI-1447, Y-27632, CCG-1423, GGTI-DU40, GGTI-297, AR9281, TPPU, AM-1172, JNJ 1661010, PF-3845, zafirlukast, montelukast, zileuton, or a combination thereof.

In some embodiments, the additional therapeutic agent is provided in a formulation comprising the additional therapeutic agent and a pharmaceutically acceptable carrier or vehicle. In some embodiments, the formulation is suitable for administration by inhalation. In some embodiments, the formulation is suitable for administration orally or by injection.

The additional therapeutic agent may treat the same disease, disorder, or symptoms as a statin, or may treat different symptoms of the same disease or disorder. Combinations of one or more statins with one or more additional therapeutic agents in some cases exhibit additive effects, in which the degree of response due to the combination formulation is substantially the same as the sum of the degree of response from each agent when administered alone. Combinations can also produce sub-additive effects, in which the combination produces a degree of response that is less than the sum of the degree of responses from each agent when administered alone (but still greater than the response produced by either agent alone), or synergistic effects, in which the combination produces a degree of response that is greater than the sum of the degree of responses from each agent when administered alone. Thus, combinations of one or more statins and one or more additional therapeutic agents can be used to achieve a greater response while administering a given dose, to achieve the same response while administering a reduced dose, or any combination thereof.

If the degree of effect produced by the combination is greater than the degree desired or required, the dose of one or both agents can be reduced until the desired degree of effect is reached. The amount of dose reduction will not necessarily be the same amount or percentage for each agent. This can be used to reduce side effects, or minimize the probability of encountering side effects. Thus, the dose of one or more agents in a synergistic combination formulation may be reduced to a level that would be insufficient or sub-therapeutic when administered alone or as part of a non-synergistic combination formulation, but is sufficient for therapeutic use when administered as part of the synergistic combination formulation. The sub-therapeutic dose of an agent in a synergistic combination formulation can be about 90%, 80%, 75%, 70%, 65%, 60%, 55%, 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% of the effective dose of the agent when administered by inhalation as part of a non-synergistic formulation according to the present disclosure.

In some systems, the administration of an inhaled statin potentiates the effect of an additional therapeutic agent that is administered at a given time period later, and provides a greater therapeutic effect than either the statin or the additional therapeutic agent alone. In some systems, the administration of an inhaled statin potentiates an effect of an additional therapeutic agent that is other than relaxation of ASM. In some systems, the additional therapeutic agent is administered later than the statin. In some embodiments, the time period is at least about 1, 2, 3, 4, 5, 6, 7, 8, 9, 10, 11, 12, 13, 14, 15, 16, 17, 18, 19, 20, 21, 22, 23, or 24 hours, or about 1, 2, or 3 days. In some embodiments, the time period is no more than about 72, 48, 36, 24, 23, 22, 21, 20, 19, 18, 17, 16, 15, 14, 13, 12, 11, 10, 9, 8, 7, or 6 hours.

In some systems, the inhaled statin potentiates, or increases, an anti-inflammatory effect of an additional therapeutic agent. In some embodiments, the degree of potentiation is a factor of about 1.1, 1.2, 1.3, 1.4, 1.5, 1.6, 1.7, 1.8, 2.0, 2.5, 3.0, 3.5, 4.0, 5, 6, 7, 8, 9, 10, 12, 15, 17, 20, 25, 30, 35, 40, 45, 50, 75, or 100-fold times the effect of an additional therapeutic agent at the dose administered. In some embodiments, the synergistic therapeutic effect is 10%, 15%, 20%, 25%, 30%, 35%, 40%, 45%, 50%, 55%, 60%, 65%, 70%, 75%, 80%, 85%, 90%, 95%, 100%, 110%, 120%, 125%, 130%, 140%, 150%, 160%, 170%, 180%, 190%, 200%, 225%, 250%, 275%, 300%, 350%, 400%, 450%, 500%, 600%, 700%, 800%, 900%, or 1,000% greater than the effect produced by the sum of the agents of the combination when those agents are administered alone at the same dose that is present in the combination.

In some embodiments, the effect is an anti-inflammatory effect. In some embodiments, the additional therapeutic agent is a β2-agonist or an anti-inflammatory corticosteroid. In some embodiments, the additional therapeutic agent is a β2-agonist. In some embodiments, the β2-agonist is albuterol, arformoterol, buphenine, clenbuterol, bopexamine, epinephrine, fenoterol, formoterol, isoetarine, isoproterenol, orciprenaline, levoalbutamol, levalbuterol, pirbuterol, procaterol, ritodrine, albuterol, salmeterol, terbutaline, arbutamine, befunolol, bromoacetylalprenololmenthane, broxaterol, cimaterol, cirazoline, etilefrine, hexoprenaline, higenamine, isoxsuprine, mabuterol, methoxyphenamine, oxyfedrine, ractopamine, reproterol, rimiterol, tretoquinol, tulobuterol, zilpaterol, or zintero, or a combination thereof. In some embodiments, the β2-agonist is albuterol or isoproterenol.

In some embodiments, the additional therapeutic agent is a corticosteroid. In some embodiments, the corticosteroid is beclomethasone, fluticasone, budesonide, mometasone, flunisolide, alclometasone, beclometasone, betamethasone, clobetasol, clobetasone, clocortolone, desoximetasone, dexamethasone, diflorasone, difluocortolone, flurclorolone, flumetasone, fluocortin, fluocortolone, fluprednidene, fluticasone, fluticasone furoate, halometasone, meprednisone, mometasone, mometasone furoate, paramethasone, prednylidene, rimexolone, ulobetasol, amcinonide, ciclesonide, deflazacort, desonide, formocortal, fluclorolone acetonide, fludroxycortide, fluocinolone acetonide, fluocinonide, halcinonide, or triamcinolone acetonide, or a combination thereof.

### Methods of Treatment

### (a) Administration by Inhalation

The methods of treatment of the disclosure are based on the administration of suitable statins by inhalation. The methods, formulations, and systems of the disclosure treat lung diseases that are not directly caused by inflammation, thus providing therapies for diseases that are not effectively or completely treated with existing therapeutic agents. Additionally, the methods, formulations, and systems of the disclosure potentiate the activity of other therapeutic agents, such as β2-agonists and corticosteroids, increasing the activity of the other therapeutic agents (which can include anti-inflammatory activity). Administration by inhalation has the advantages of (a) direct contact with the respiratory airways, (b) avoidance of first-pass hepatic metabolism, and (c) avoidance of injection (J. L. Rau, Resp Care (2005) 50(3):367-82; M. Ibrahim et al., Med Dev Evidence Res (2015) 8:131-39). Because the drug is not subject to first-pass metabolism, and is administered locally to the lungs rather than systemically to the entire body, the doses for inhaled drugs are often smaller than the amount that would be administered orally. Further, methods, formulations, and systems of the disclosure that potentiate the activity of other therapeutic agents can provide treatment with greater activity, or match existing treatment with a lower dose of the other therapeutic agent, or a combination thereof.

As set forth herein, formulations of the disclosure are administered with the aid of an inhalation device (“inhaler”), which can be a nebulizer, pMDI, DPI, or other device capable of conveying the formulation into the lower airways. The frequency of administration will depend on the clearance rate of the statin and/or additional therapeutic agent from the subject's lungs. In some embodiments, a statin formulation is administered no more than 8, 7, 6, 5, 4, 3, 2, or once per day, or no more than once every 2, 3, 4, 5, 6, or 7 days. In some embodiments, a statin formulation is administered at least once every 4, 3, or 2 days, or at least 1, 2, 3, 4, 5, or 6 times per day.

In the methods of the disclosure, the therapeutic composition is administered directly to the lungs, and thus does not undergo first pass metabolism in the liver. As a result, the active agents in the formulation are not diluted across the subject's entire body, and are not metabolized by the liver, such that a smaller amount is required reach a therapeutic concentration in the subject's airways than would be required with conventional, oral administration. The therapeutically effective amount will depend on the condition to be treated, the severity of the condition, the general health and state of the subject, and the particular statin(s) (and/or isomer(s), enantiomer(s), and/or diastereomer(s)) selected. Thus, a therapeutically effective amount of a statin in the practice of the disclosure may be as low as about 0.005 μg, about 0.008 μg, about 0.01 μg, about 0.05 μg, about 0.08 μg, about 0.1 μg, about 0.5 μg, about 0.8 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 14 μg, about 15 μg, about 16 μg, about 18 μg, or about 20 μg. A therapeutically effective amount of a statin in the practice of the disclosure may be as high as about 40 mg, 20 mg, 18 mg, 15 mg, 12 mg, 10 mg, 9 mg, 8 mg, 7 mg, 6 mg, 5 mg, 4 mg, 3 mg, 2 mg, or 1 mg.

In some embodiments, the therapeutically effective amount of the statin is at least about 0.005 μg/kg, about 0.008 μg/kg, about 0.01 μg/kg, about 0.05 μg/kg, about 0.08 μg/kg, about 0.1 μg/kg, about 0.5 μg/kg, about 0.8 μg/kg, about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 11 μg/kg, about 12 μg/kg, about 14 μg/kg, about 15 μg/kg, about 16 μg/kg, about 18 μg/kg, or about 20 μg/kg. In some embodiments, the therapeutically effective amount of the statin is no higher than about 40 mg/kg, 20 mg/kg, 18 mg/kg, 15 mg/kg, 12 mg/kg, 10 mg/kg, 9 mg/kg, 8 mg/kg, 7 mg/kg, 6 mg/kg, 5 mg/kg, 4 mg/kg, 3 mg/kg, 2 mg/kg, or 1 mg/kg.

### (b) Reducing Airway Smooth Muscle Contraction

Airways in the lungs of mammals are lined with airway smooth muscle (ASM), covered by a thin layer of airway epithelial cells (AEC). ASM is an involuntary muscle tissue that contributes to regulation of pulmonary function by contraction and relaxation, which controls the diameter of the airways (bronchi and bronchioles). In some disorders, ASM contracts inappropriately, narrowing airways and increasing the effort required to breathe. Conventional therapies seek to inhibit the contraction by blocking or reducing inflammatory stimuli. In the methods of the disclosure, ASM is caused to relax by administering a formulation of the disclosure directly to the airways. Without intending to be bound by any particular theory, the method of the disclosure reduces the contractile responsiveness, reduces the force exerted by ASM, and inhibits hyperproliferation of ASM (which also causes narrowing of airways). In some embodiments, the present disclosure provides a method of reducing ASM contraction, by administering an effective amount of a formulation of the disclosure by inhalation.

In some embodiments, the present disclosure provides a method of reducing airway smooth muscle (ASM) contraction in a subject by administering a therapeutically effective amount of a statin (or an isomer, enantiomer, or diastereomer thereof) with a pharmaceutically acceptable carrier to the subject by inhalation. Relaxation of ASM reduces obstruction to breathing, and can increase resting lung capacity. Thus, the methods of the disclosure are useful in the treatment of lung diseases that are characterized by, or otherwise include, obstruction of a subject's airways, such as bronchoconstriction or bronchospasm as is inherent in asthma, COPD, ACOS, cystic fibrosis, bronchiectasis, idiopathic pulmonary fibrosis, alpha-1 antitrypsin deficiency (AATD), and the like. Additional lung diseases and disorders which can have direct or indirect effects on the lung airways (and thus can be ameliorated with methods of the disclosure) include interstitial lung diseases (ILD) such as, without limitation, lung fibrosis, idiopathic pulmonary fibrosis (IPF), desquamative interstitial pneumonia (DIP), acute interstitial pneumonia (AIP), nonspecific interstitial pneumonia (NSIP), respiratory bronchiolitis-associated interstitial lung disease (RB-ILD), cryptogenic organizing pneumonia (COP), lymphoid interstitial pneumonia (LIP). Additional diseases and disorders that may involve the lungs, and that can benefit from the methods of the disclosure, include sarcoidosis, rheumatoid arthritis, systemic lupus erythematosus (SLE), systemic sclerosis, polymyositis, dermatomyositis, antisynthetase syndrome, pulmonary infections, hypersensitivity pneumonitis, and reactions to acute or chronic exposure to foreign substances such as asbestos, beryllium, silica, industrial chemicals and irritant particles. In such additional lung diseases and disorders, symptoms such as increased airway wall thickness (see, e.g., J. M. Oldham, Ann Am Thorac Soc (2019) 16(4):432-33; E. R. Miller et al., Ann Am Thorac Soc (2019) 16(4):447-54) may be ameliorated by the administration of statins by inhalation.

ASM relaxation can be determined in vitro by, for example without limitation, methods for measuring cellular forces, and by measurement of airway lumen changes in lung tissue samples. For example, ASM contraction forces can be measured using the techniques described in R. Rokhzan et al., Lab Invest (2019) 99(1):138-45, in which the displacement of fluorescent beads on a substrate of known stiffness is measured. The measurement of airway lumen changes in lung tissue samples can be accomplished using the methods described in K. R. Patel et al., FASEB J (2017) 31(10):4335-46, in which airway diameters are measured in precision cut lung slices.

ASM relaxation can be determined in vivo by standard pulmonary function tests, for example without limitation, spirometry and lung volume determination. Spirometry is the measurement of the breath, including the volume of air and/or the rate at which it is inhaled or exhaled. Typical measurements include the forced volume vital capacity (FCV), in which the subject takes the deepest breath he or she can, and exhales it into a spirometer as hard and long as possible; forced expiratory volume at 1 second (FEV₁), which is a measurement of how much air the subject can exhale within 1 second; maximum voluntary ventilation (MVV); forced expiratory flow (FEF), and the like. Other parameters include lung volume, lung capacity, vital capacity, and others, which are generally also measured by spirometry. An increased value for any of the foregoing is indicative of ASM relaxation, in that ASM relaxation reduces obstruction and can increase the lung vital capacity. The increase in any of these values can be measured against the subject's measurement prior to treatment, and/or against a standard predicted value for a subject of similar height, weight, and gender. Methods of the disclosure induce ASM relaxation by at least 5, 10, 15, 20, 25, 30, 35, 40, 45, 50, 55, 60, 65, 70, 75, 80, 85, 90, 95, 100, 105, 110, 120, 130, 140, or 150%. The upper limit to ASM relaxation is about 1,000, 900, 800, 700, 600, 500, 400, 300, 200, 100, 90, 80, 70, 60, or 50%.

A therapeutically effective amount of a statin for inducing ASM relaxation may be as low as about 0.005 μg, about 0.008 μg, about 0.01 μg, about 0.05 μg, about 0.08 μg, about 0.1 μg, about 0.5 μg, about 0.8 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 14 μg, about 15 μg, about 16 μg, about 18 μg, or about 20 μg. A therapeutically effective amount of a statin for inducing ASM relaxation may be as high as about 40 mg, 20 mg, 18 mg, 15 mg, 12 mg, 10 mg, 9 mg, 8 mg, 7 mg, 6 mg, 5 mg, 4 mg, 3 mg, 2 mg, or 1 mg.

### (c) Reducing Bronchospasm

Bronchospasm is the sudden contraction or constriction of the bronchioles, typically in response to an inflammatory stimulus, for example, exposure to an allergen, mast cell degranulation, and administration of certain drugs. It occurs in asthma, chronic bronchitis, and anaphylaxis, and can be life-threatening. Bronchospasm is characteristic of asthma exacerbations (“asthma attacks”) Administration of formulations of the disclosure by inhalation treats bronchospasm (and asthma exacerbations) by reducing airway hyper-reactivity and hyperresponsiveness, making bronchospasm less likely to occur, and by reducing the ASM contractile force, making bronchospasm less severe if it does occur. In some embodiments, the present disclosure provides a method of reducing bronchospasm, by administering an effective amount of a formulation of the disclosure by inhalation.

Bronchospasm can include, for example, post-infectious bronchospasm due to viral, bacterial, fungal, and/or mycobacterial infection; airway edema due to congestive heart failure; airway edema due to pulmonary edema; airway edema due to cardiogenic pulmonary edema; airway edema due to non-cardiogenic pulmonary edema; bronchiolitis due to airway edema; bronchiectasis due to anatomic distortions rather than inflammation; foreign body aspiration; aspiration of food, liquids, and/or gastric contents; gastro-esophageal reflux disease; lung cancer or metastatic cancer to the lung causing local edema and bronchospasm; pulmonary embolism (which can release local factors that cause wheezing due to bronchospasm); airway trauma, including surgery; anaphylaxis and anaphylactoid reactions; neurally mediated cough and/or bronchospasm; inhalation injury-associated bronchospasm; endocrine dysfunction associated bronchospasm; paraneoplastic syndrome-associated bronchospasm

A therapeutically effective amount of a statin for the reduction of bronchospasm may be as low as about 0.005 μg, about 0.008 μg, about 0.01 μg, about 0.05 μg, about 0.08 μg, about 0.1 μg, about 0.5 μg, about 0.8 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 14 μg, about 15 μg, about 16 μg, about 18 μg, or about 20 μg. A therapeutically effective amount of a statin may be as high as about 40 mg, 20 mg, 18 mg, 15 mg, 12 mg, 10 mg, 9 mg, 8 mg, 7 mg, 6 mg, 5 mg, 4 mg, 3 mg, 2 mg, or 1 mg.

Reduction of bronchospasm can be measured by counting the number of bronchospasm events (for example, asthma exacerbations) over a period of time, and comparing this frequency to the frequency observed prior to treatment. Reduction of bronchospasm can also be measured by measuring the subject's pulmonary function (for example, FEV₁) prior to challenge, then administering a dose (or series of increasing doses) of nebulized methacholine or histamine, then measuring the subject's pulmonary function again. Having obtained a baseline value, the subject is treated by inhalation of a formulation of the disclosure, and after an appropriate amount of time, is subjected to the challenge again. The challenge can be administered about 1, 2, 4, 6, 8, 10, 12, 14, 16, 18, 20, or 24 or more hours after inhalation of the formulation of the disclosure. The reduction in bronchoconstriction is determined by the improvement in pulmonary function after challenge, for example by comparing the FEV₁ after administration and challenge against the FEV₁ before administration but after challenge. Methods of the disclosure reduce bronchospasm by at least 5, 10, 15, 20, 25, 30, 35, 40, 45, 50, 55, 60, 65, 70, 75, 80, 85, 90, 95, 100, 105, 110, 120, 130, 140, or 150%. The upper limit to bronchospasm reduction is about 1,000, 900, 800, 700, 600, 500, 400, 300, 200, 100, 90, 80, 70, 60, or 50%.

### (d) Reduction of Bronchoconstriction and Mucus Accumulation

Some airway diseases result in chronically constricted or obstructed bronchi and bronchioles, which further causes an accumulation of mucus. This is characteristic of diseases such as emphysema, asthma, COPD, cystic fibrosis, allergen-induced bronchoconstriction, and exercise-induced bronchoconstriction (also known as exercise-induced asthma). Administering a formulation of the disclosure by inhalation treats bronchoconstriction and mucus accumulation by relaxing the ASM and dilating the airways. In some embodiments, the present disclosure provides a method of reducing bronchoconstriction and/or mucus accumulation, by administering an effective amount of a formulation of the disclosure by inhalation.

Although bronchoconstriction, bronchospasm, and ASM relaxation are distinct indications, improvement in each can be measured by tests of pulmonary function, such as spirometry, as set forth above. Methods of the disclosure reduce bronchoconstriction by at least 5, 10, 15, 20, 25, 30, 35, 40, 45, 50, 55, 60, 65, 70, 75, 80, 85, 90, 95, 100, 105, 110, 120, 130, 140, or 150%. The upper limit to bronchoconstriction reduction is about 1,000, 900, 800, 700, 600, 500, 400, 300, 200, 100, 90, 80, 70, 60, or 50%. A therapeutically effective amount of a statin for reducing bronchoconstriction may be as low as about 0.005 μg, about 0.008 μg, about 0.01 μg, about 0.05 μg, about 0.08 μg, about 0.1 μg, about 0.5 μg, about 0.8 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 14 μg, about 15 μg, about 16 μg, about 18 μg, or about 20 μg. A therapeutically effective amount of a statin for reducing bronchoconstriction may be as high as about 40 mg, 20 mg, 18 mg, 15 mg, 12 mg, 10 mg, 9 mg, 8 mg, 7 mg, 6 mg, 5 mg, 4 mg, 3 mg, 2 mg, or 1 mg.

### (e) Reduction of Bronchial Hyperresponsiveness

Bronchial hyperresponsiveness (also called BH, airway hyperresponsiveness, AHR, or airway hyperreactivity) is a condition in which bronchospasm is easily triggered or induced. The methods of the disclosure, administering a formulation of the disclosure by inhalation, reduce BH by relaxing ASM and reducing the sensitivity of the ASM. In some embodiments, the present disclosure provides a method of reducing bronchial hyperresponsiveness, by administering an effective amount of a formulation of the disclosure by inhalation.

As BH is an airway disorder, it is also generally measured by spirometry and other measures of pulmonary function. For example, one can measure a subject's FEF after administration of the formulation and challenge with a triggering substance such as nebulized methacholine or histamine, and comparing this to the FEF after challenge with the same quantity of triggering substance but without administration of the formulation of the disclosure. The challenge can be administered about 1, 2, 4, 6, 8, 10, 12, 14, 16, 18, 20, or 24 or more hours after inhalation of the formulation of the disclosure. Methods of the disclosure reduce BH by at least 5, 10, 15, 20, 25, 30, 35, 40, 45, 50, 55, 60, 65, 70, 75, 80, 85, 90, 95, 100, 105, 110, 120, 130, 140, or 150%. The upper limit to BH reduction is about 1,000, 900, 800, 700, 600, 500, 400, 300, 200, 100, 90, 80, 70, 60, or 50%. A therapeutically effective amount of a statin for reducing BH may be as low as about 0.005 μg, about 0.008 μg, about 0.01 μg, about 0.05 μg, about 0.08 μg, about 0.1 μg, about 0.5 μg, about 0.8 μg, about 1 μg, about 2 μg, about 3 μg, about 4 μg, about 5μg, about 6 μg, about 7 μg, about 8 μg, about 9 μg, about 10 μg, about 11 μg, about 12 μg, about 14 μg, about 15 μg, about 16 μg, about 18 μg, or about 20 μg. A therapeutically effective amount of a statin for reducing BH may be as high as about 40 mg, 20 mg, 18 mg, 15 mg, 12 mg, 10 mg, 9 mg, 8 mg, 7 mg, 6 mg, 5 mg, 4 mg, 3 mg, 2 mg, or 1 mg.

### (f) Increasing Stretch-Induced ASM Relaxation

ASM can be induced to relax somewhat by taking a deep breath (“deep inspiration”), which stretches the ASM. The methods of the disclosure potentiate, increase, and/or extend this breath-induced ASM relaxation (also referred to as breath-induced bronchodilation, or deep inspiration bronchodilation, DIB). In some embodiments, the present disclosure provides a method of potentiating deep breath-induced ASM relaxation, by administering an effective amount of a formulation of the disclosure by inhalation.

As DIB is an airway function, it is also generally measured by spirometry and other measures of pulmonary function. For example, one can measure a subject's vital capacity (VC) after administration of the formulation, and comparing this to the VC without administration of the formulation of the disclosure: increased VC correlates with potentiated breath-induced ASM relaxation. The VC can be measured about 1, 2, 4, 6, 8, 10, 12, 14, 16, 18, 20, or 24 or more hours after inhalation of the formulation of the disclosure. Methods of the disclosure increase DIB by at least 5, 10, 15, 20, 25, 30, 35, 40, 45, 50, 55, 60, 65, 70, 75, 80, 85, 90, 95, 100, 105, 110, 120, 130, 140, or 150%. The upper limit to DIB increase is about 1,000, 900, 800, 700, 600, 500, 400, 300, 200, 100, 90, 80, 70, 60, or 50%. A therapeutically effective amount of a statin for potentiating DIB may be as low as about 0.005 μg, about 0.008 μg, about 0.01 μg, about 0.05 μg, about 0.08 μg, about 0.1 μg, about 0.5 μg, about 0.8 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 14 μg, about 15 μg, about 16 μg, about 18 μg, or about 20 μg. A therapeutically effective amount of a statin for potentiating DIB may be as high as about 40 mg, 20 mg, 18 mg, 15 mg, 12 mg, 10 mg, 9 mg, 8 mg, 7 mg, 6 mg, 5 mg, 4 mg, 3 mg, 2 mg, or 1 mg.

### (g) Reduction of Corticosteroid Use

Inhaled corticosteroids (ICS) are commonly used in the treatment of severe asthma. However, chronic use of ICS can also have significant side effects, such as dysphonia, decreased bone density, skin thinning and bruising, cataracts, and others. The methods of the disclosure reduce and treat asthma and other airway diseases, reducing the need for a subject to take ICS. In some embodiments, the present disclosure provides a method of reducing the need for ICS, by administering an effective amount of a formulation of the disclosure by inhalation.

The reduction in need for ICS can be determined by measuring a subject's pulmonary function, for example by spirometry, while using ICS, placing the subject on a treatment regime of regular administration of a formulation of the disclosure by inhalation, and gradually reducing the dosage or frequency of ICS usage to the point that the subject's pulmonary function is reduced to the subject's pulmonary function before beginning the inhaled statin treatment (if that point can be reached). Methods of the disclosure reduce ICS need by at least 5, 10, 15, 20, 25, 30, 35, 40, 45, 50, 55, 60, 65, 70, 75, 80, 85, 90, 95, 100% (i.e., at which point the statin completely replaced the ICS). The upper limit to ICS need reduction is about 100, 90, 80, 70, 60, 50, 45, 40, 35, 30, or 25%. A therapeutically effective amount of a statin for reducing ICS need may be as low as about 0.005 μg, about 0.008 μg, about 0.01 μg, about 0.05 μg, about 0.08 μg, about 0.1 μg, about 0.5 μg, about 0.8 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 14 μg, about 15 μg, about 16 μg, about 18 μg, or about 20 μg. A therapeutically effective amount of a statin for reducing ICS need may be as high as about 40 mg, 20 mg, 18 mg, 15 mg, 12 mg, 10 mg, 9 mg, 8 mg, 7 mg, 6 mg, 5 mg, 4 mg, 3 mg, 2 mg, or 1 mg.

### (h) Method of Maintaining or Improving Pulmonary Function

Pulmonary function varies from subject to subject, and is generally increased (higher than average) in athletes, mountain climbers, and subjects who live at high altitude. Improved pulmonary function can be measured by spirometry, and often manifests as increased VC or lung capacity (LC). Methods of the disclosure are also useful for improving pulmonary function in healthy subjects, as well as subjects having an airway disorder. For example, it is advantageous to have increased pulmonary function for, for example, athletes, mountain climbers, soldiers, wind musicians, and orators. Additionally, subjects can maintain a given degree of pulmonary function by the methods of the disclosure, for example during a period in which exercise is not possible due to injury. Improved (or maintained) pulmonary function can be measured by spirometry as, for example without limitation, increased VC or lung capacity (LC). In some embodiments, the present disclosure provides a method of maintaining or improving pulmonary function, by administering an effective amount of a formulation of the disclosure by inhalation.

Methods of the disclosure increase pulmonary function by at least 5, 10, 15, 20, 25, 30, 35, 40, 45, 50, 55, 60, 65, 70, 75, 80, 85, 90, 95, 100, 105, 110, 120, 130, 140, or 150% (depending on the starting state of the subject). The upper limit to pulmonary function increase is about 500, 400, 300, 200, 100, 90, 80, 70, 60, 50, 45, 40, 35, 30, 25, or 20%. A therapeutically effective amount of a statin for increasing pulmonary function may be as low as about 0.005 μg, about 0.008 μg, about 0.01 μg, about 0.05 μg, about 0.08 μg, about 0.1 μg, about 0.5 μg, about 0.8 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 14 μg, about 15 μg, about 16 μg, about 18 μg, or about 20 μg. A therapeutically effective amount of a statin for increasing pulmonary function may be as high as about 40 mg, 20 mg, 18 mg, 15 mg, 12 mg, 10 mg, 9 mg, 8 mg, 7 mg, 6 mg, 5 mg, 4 mg, 3 mg, 2 mg, or 1 mg.

Maintaining pulmonary function in some cases requires less statin than increasing pulmonary function, for example the dose may be as low as about 0.001, 0.005, 0.008, 0.01, 0.05, 0.08, 0.1, 0.5, 0.8, 1, 2, 3, 4, 5, 6, 7, 8, 9, 10, 11, 12, 14, 15, 16, 18, or 20 μg. A therapeutically effective amount of a statin for increasing pulmonary function may be as high as about 20 mg, 18 mg, 15 mg, 12 mg, 10 mg, 9 mg, 8 mg, 7 mg, 6 mg, 5 mg, 4 mg, 3 mg, 2 mg, or 1 mg.

### (i) Method of Reducing ASM Proliferation

COPD, ACOS, cystic fibrosis, and chronic asthma can all exhibit narrowing of airways due to hyperproliferation and thickening of ASM associated with pathological airway remodeling, apart from any bronchoconstriction. Methods of the disclosure also reduce ASM proliferation, thus treating such disorders. Inhibition of ASM proliferation can be measured by spirometry, as maintained VC or lung capacity (LC), or by imaging methods such as X-ray or MRI. In some embodiments, the present disclosure provides a method of reducing ASM proliferation, by administering an effective amount of a formulation of the disclosure by inhalation.

Methods of the disclosure reduce ASM proliferation by at least 5, 10, 15, 20, 25, 30, 35, 40, 45, 50, 55, 60, 65, 70, 75, 80, 85, 90, 95, 100% (i.e., at which point no ASM proliferation is observed). The upper limit to ASM proliferation reduction is about 100, 90, 80, 70, 60, 50, 45, 40, 35, 30, or 25%. A therapeutically effective amount of a statin for reducing ASM proliferation may be as low as about 0.005 μg, about 0.008 μg, about 0.01 μg, about 0.05 μg, about 0.08 μg, about 0.1 μg, about 0.5 μg, about 0.8 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 14 μg, about 15 μg, about 16 μg, about 18 μg, or about 20 μg. A therapeutically effective amount of a statin for reducing ASM proliferation may be as high as about 40 mg, 20 mg, 18 mg, 15 mg, 12 mg, 10 mg, 9 mg, 8 mg, 7 mg, 6 mg, 5 mg, 4 mg, 3 mg, 2 mg, or 1 mg.

### (j) Method of Treating Interstitial Lung Disease

Interstitial lung disease (ILD) directly affects lung tissue outside the airways. However, ILDs can have an effect on airways and ASM, causing one or more symptoms that can be treated by the methods of the disclosure. These symptoms can include, for example, ASM contraction, ASM hyperproliferation, and loss of lung capacity. In some embodiments, the present disclosure provides a method for aiding in the treatment of an ILD that affects the airways of a subject, by administering a formulation of the disclosure by inhalation in an amount sufficient to reduce one or more symptoms. Examples of specific ILDs are set forth above.

Reduction of one or more ILD symptoms can be measured using spirometry, such as FEV₁ and LC, and imaging techniques such as X-ray and MRI. Methods of the disclosure reduce at least one ILD symptom by at least 5, 10, 15, 20, 25, 30, 35, 40, 45, 50, 55, 60, 65, 70, 75, 80, 85, 90, 95, 100% (i.e., at which point the symptom is no longer observed). The upper limit to ILD symptom reduction is about 100, 90, 80, 70, 60, 50, 45, 40, 35, 30, or 25%. A therapeutically effective amount of a statin for reducing one or more ILD symptoms may be as low as about 0.005 μg, about 0.008 μg, about 0.01 μg, about 0.05 μg, about 0.08 μg, about 0.1 μg, about 0.5 μg, about 0.8 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 14 μg, about 15 μg, about 16 μg, about 18 μg, or about 20 μg. A therapeutically effective amount of a statin for reducing one or more ILD symptoms may be as high as about 40 mg, 20 mg, 18 mg, 15 mg, 12 mg, 10 mg, 9 mg, 8 mg, 7 mg, 6 mg, 5 mg, 4 mg, 3 mg, 2 mg, or 1 mg.

In some embodiments, the present disclosure provides a method of treating COPD in a subject. COPD can be characterized as a destruction of both small airways and parenchyma resulting in a progressive impairment in pulmonary function. The disease may be divided into two subgroups, namely chronic bronchitis and emphysema. Chronic bronchitis is characterized by mucus hypersecretion from the conducting airways, inflammation and eventual scarring of the bronchi (airway tubes). Many persons with COPD have a component of both of these conditions.

The interaction between parenchymal disease and the vasculature is often clinically evident by the observation that patients with severe COPD have mild or moderate pulmonary hypertension at rest. Histopathologically and microscopically, the pulmonary vasculature in COPD is typically characterized by initial thickening with smooth muscle deposition as well as a loss of both alveolar septal structures and microvasculature. It has also been observed that both alveolar septal and endothelial cells undergo apoptosis in COPD.

The presenting symptoms for COPD are typically breathlessness accompanied by a decline in FEV₁ (i.e., forced expiratory volume in 1 second) and/or forced vital capacity (FVC). COPD patients have difficulty breathing because they develop smaller, inflamed air passageways and have partially destroyed alveoli. Chronic bronchitis can also be diagnosed by asking the patient whether they have a “productive cough”, i.e., one that yields sputum. The patients' symptoms are cough and expectoration of sputum. Chronic bronchitis can lead to more frequent and severe respiratory infections, narrowing and plugging of the bronchi, difficult breathing, and disability.

In some embodiments, the present disclosure provides a method of treating emphysema in a subject. Emphysema is a chronic lung disease which affects the alveoli and/or the ends of the smallest bronchi. The condition is characterized by destructive changes and enlargement of the alveoli (air sacs) within the lungs. The lung loses its elasticity and therefore these areas of the lungs become enlarged. These enlarged areas trap stale air and do not effectively exchange it with fresh air. This results in difficult breathing and may result in insufficient oxygen being delivered to the blood. The predominant symptom in patients with emphysema is shortness of breath.

In some embodiments, the present disclosure provides a method of treating a subject whose symptoms are poorly controlled by his or her current medication, by administering a formulation of the disclosure instead of, or in combination with, the subject's current medication.

In the practice of the methods of the disclosure, lung diseases are treated by reducing airway smooth muscle contraction in a subject by administering a formulation to a subject in need thereof by inhalation, wherein the formulation contains a therapeutically effective amount of a statin, or an isomer, enantiomer, or diastereomer thereof, and a pharmaceutically acceptable carrier.

### EXAMPLES

The following examples are provided for guidance, and are not intended to limit the scope of the claims herein.

Primary human airway smooth muscle (ASM) cells derived from both non-asthmatic and asthmatic donors were obtained from the Gift of Hope Organ and Tissue Donor Network.

These cells have been well-characterized previously (see for example H. Yoshie et al., Biophys J (2018) 114(9):2194-99). All measurements were performed using cells at passage 5-8 from three non-asthmatic donors. Cells were grown on either 10% serum-containing F12 complete media, or serum-deprived media supplemented with insulin, transferrin, and selenium (Corning, Tewksbury, MA).

To enhance cellular uptake and predictable cellular relaxation property simvastatin was activated by alkaline hydrolysis to chemically convert simvastatin lactone to simvastatin acid (SA). In vivo, hydrolysis can also occur naturally inside cells via lactonases, paraoxonases, alkaline hydrolases, and carboxylesterases. Simvastatin was activated by opening its lactone ring, using the protocol provided by Merck. Briefly, 8 mg of simvastatin (0.019 mM) is dissolved in 0.2 mL of 100% ethanol, with subsequent addition of 0.3 mL of 0.1 N NaOH. The solution is then heated at 50° C. for 2 hours in a sand bath, then neutralized with HCl to a pH of 7.2 (C. C. Ghosh et al., Crit Care Med (2015) 43(7):e230-40).

### Example 1: Relaxation of ASM Contractile Forces

The following experiments demonstrate that statins can cause relaxation of ASM in resting, non-stimulated cells.

Contractile Force Screening: Human ASM cells were grown to confluence in 96-well plates on custom NuSil™ 8100 elastic substrates (Avantor, Inc., Radnor, PA) (R. Rokhzan et al., Lab Invest (2019) 99(1):138-45). Fluorescent beads (diameter ~400 nm) were embedded in the substrate surface to enable traction force calculations based on their displacements. To measure tractions, an inverted epi-fluorescence microscope (DMI 6000B, Leica Inc., Germany) equipped with a heated chamber (37°C), a monochrome camera (Leica DFC365 FX), and a motorized stage was used. Spatial images were recorded of substrate-embedded fluorescent beads at 10× magnification. Based on the bead displacements (resolution ~15 μm) relative to a cell-free model, together with knowledge of substrate stiffness and thickness, tractions were computed using the approach of Fourier Transform Traction Cytometry (B. Yeganeh et al., Pharmacol Ther (2014) 143(1):87-110), modified to the case of cell monolayers (E. J. Whalen et al., Cell (2007) 129(3):511-22). From each traction map, the root-mean-squared traction (RMST) value and strain energy were calculated and reported as a measure of normal contraction in the monolayer. On a well-by-well basis, the ratio of the strain energy after vs. before treatment was computed, and all values of a given treatment group were normalized to the mean of each treatment group.

Comparison of Statins: To determine effects of statins on contractile machinery independent of inflammation, primary ASM cells were grown on bead embedded NuSil™ (3 kPa stiffness) and treated with simvastatin-acid (SA), rosuvastatin, pravastatin, and pitavastatin (each 1 μM), with or without mevalonate (MA, 100 μM). MA is the immediate product of HMGCR action on HMB-CoA, so addition of MA would reverse the effect of statin inhibition of HMGCR. Contractile force screening (CFS) demonstrated that statins directly relax human ASM at the basal state (FIG. 1A). The lipophilic statins are the most potent (simvastatin<pitavastatin). Conversely, the other hydrophilic statins such as rosuvastatin and pravastatin had little to no effect on ASM cell relaxation, confirming differential statin drug effects, presumably due to differences in lipophilicity. Co-administration of MA with statins abrogated the relaxation effects of statins on ASM, suggesting that ASM tone is dependent on MA or the MA pathway, and confirming an MA-dependent mechanism for statin-induced relaxation in ASM cells (FIG. 1B).

Simvastatin-acid (SA), atorvastatin, pravastatin, and pitavastatin were examined with CFS to determine the dose response of the ASM relaxation effect. Simvastatin-acid (SA), atorvastatin, pravastatin, and pitavastatin were each added to primary ASM cells as described above, at 0, 0.08, 0.4, 2, and 10 μM. In a second experiment, Simvastatin-acid (SA), rosuvastatin, pravastatin, and pitavastatin were each added to primary ASM cells as described above, at 0, 1, and 10 μM. These dose-response experiments further verified that the inhibitory potency of statins on ASM contraction varies according to lipophilicity (simvastatin ~pitavastatin>atorvastatin>>pravastatin), where the most lipophilic statins simvastatin and pitavastatin had the most significant effect, as compared to the less lipophilic atorvastatin and the hydrophilic pravastatin (FIGS. 1C, 1D).

Next, the dose-dependent effects of SA and pitavastatin on primary ASM cells obtained from three different human donors were compared, and showed that both SA and pitavastatin dose-dependently relax ASM (FIG. 3).

Cellular force measurements were performed in a custom 96-well plate (substrate stiffness=3 kPa) prepared using the method of contractile force screening implemented using an inverted fluorescence microscope (10× microscope objective, Leica DMI6000 B, Leica Microsystems, Buffalo Grove, IL). From each ASM force map, the strain energy (i.e., the energy that is imparted to the substrate by the contractile cells, in pJ) was computed, to represent the average cellular contraction.

Well-defined biaxial stretch (4 sec duration, 10% magnitude) was imposed using the method of cell mapping rheometry. The strain energy from each ASM force map was computed as a metric of average cellular contraction, and reported as fold changes to the pre-stretch baseline value.

Using the method of contractile force screening, it was determined that the lipophilic statins, pitavastatin and simvastatin inhibited ASM contraction, while the hydrophilic statin, pravastatin did not (FIG. 10A). Further, when compared to simvastatin, pitavastatin's relaxation effect was more significant at 24 hrs (FIG. 10B). The force inhibitory effect of pitavastatin was reversible after cessation of treatment (FIG. 10D).

Pitavastatin did not demonstrate any cellular (FIG. 10E) or lung tissue toxicity (FIG. 10F). To establish clinical relevance, pitavastatin's effects in multiple ASM cell lines obtained from both non-asthmatic and asthmatic human donor lungs were evaluated. Additionally, asthmatic ASM was more contractile than non-asthmatic ASM (FIG. 11A). Regardless of donor (asthmatic donors: D1-D3; non-asthmatic donors: D4-D6) or disease status (non-asthmatic vs. asthmatic), pitavastatin dose-dependently inhibited ASM contraction (FIG. 11A).

Lack of apoptosis: Some studies have shown that statins at high enough doses can reduce the viability of vascular smooth muscle cells, epithelial cells, and endothelial cells (S. Ghavami et al., Biochim Biophys Acta (2014) 1843(7):1259-71; T. P. Miettinen et al., Cell Rep (2015) 13(11):2610-20). To ascertain that statin-mediated cellular relaxation is independent of apoptosis or loss of cell viability, cell-based apoptosis/necrosis assays were performed after treating ASMs with simvastatin (SA), pitavastatin, rosuvastatin, and pravastatin dose-dependently for 24 hours following the manufacturer's protocol (FIG. 2).

RealTime-Glo™ Annexin V Apoptosis and Necrosis assay was performed by growing ASM cells on 96-well white cell culture plate according to the manufacturer's instructions (Promega Inc). Cells were treated with simvastatin acid, rosuvastatin, pitavastatin, and pravastatin at doses ranging from 0.08 μM to 100 μM. In this real-time annexin V binding assay, the luminescence signal was detected for apoptosis and necrosis was detected with a fluorescence signal by using a SpectraMax® plate reader. Digoxigenin (20 μg/mL) was used as a positive control (FIG. 2).

Apoptosis was not observed in cells treated with up to 30 μM of simvastatin (treated as SA), pitavastatin, pravastatin, or up to 100 μM of rosuvastatin (FIG. 2). This suggests that lipophilic statins are well tolerated in the ASMs. This further indicates that the positive effects observed on statin-induced ASM relaxation were not due to cell toxicity or cell death.

### Example 2: Histamine-Induced Contraction

These experiments were conducted to determine the ability of statins to reduce ASM contraction induced by contact with histamine.

Cells were pretreated with pitavastatin and simvastatin acid at 0, 0.08, 0.4, 2, 10, or 50 μM for 24 hours, then challenged with histamine (10 μM). Both pitavastatin (FIG. 4A) and simvastatin (FIG. 4B) significantly reduced histamine-induced ASM contraction, in both complete and serum-starved media (FIG. 4C). In complete media, 0.08 μM pitavastatin is sufficient to inhibit histamine-mediated ASM contraction (FIG. 4A). However, to achieve a similar protection, a 5× molar excess of simvastatin (SA) was required (FIG. 4B). In serum starved media, 0.4 μM pitavastatin and 10 μM simvastatin provide a similar extent of protection from histamine-induced contraction.

Time-dependent effects of both SA and pitavastatin show that ASM relaxing effects start as early as 4 hours (FIG. 4C). Therefore, this experiment illustrates that a) pitavastatin is at least 5 times more potent than SA in prevention of histamine-induced ASM contraction, and b) availability at a nanomolar concentration of the statins in the lung may be sufficient to inhibit histamine-mediated bronchoconstriction.

A non-transplantable non-asthmatic human donor lung was obtained through the Gift of Hope/Regional Organ Bank of Illinois, and sliced as per published protocols. Briefly, the lung lobe was filled with 1.5% low melting temperature agarose (Type IX; Sigma, St. Louis, MO) in Hanks balanced salt solution (pH=7.4; Invitrogen, Carlsbad, CA), and sliced using a VT1200S vibrating blade microtome (Leica Microsystems, Bannockburn, IL) to create 250 μm thick slices. Slices were cryopreserved till the day of the experiment.

Slices were treated with pitavastatin (2 μM) for 24 hrs, before challenge with histamine (1μM). Airway constriction was measured as luminal area changes in response to increasing doses of histamine. In both sets of PCLS samples, airway lumen area was quantified from bright field images using the Fiji image analysis software.

In bronchial airways of human PCLS, pitavastatin significantly inhibited 1 μM histamine-induced airway constriction (FIG. 11C). Pitavastatin augmented stretch-induced ASM force relaxation (FIG. 12B). Strikingly, this bronchodilatory function was not conferred to the ASM by isoproterenol. Thus, pitavastatin provides novel and additive therapeutic benefit beyond existing β2-agonist bronchodilators.

### Example 3: Deep Breath Relaxation

This experiment demonstrates that pitavastatin potentiates the ASM relaxation effect of a simulated deep breath, in contrast to the β2-agonist isoproterenol.

Normal ASM was treated with pitavastatin (1 μM, 24 hrs, n=7), isoproterenol (10 μM, 30 min, n=6) or vehicle (n=7), and examined by CFS as described above. FIG. 12A shows that, as compared to the untreated controls, pre-treatment with pitavastatin significantly inhibited basal ASM contraction. Shown are contraction values normalized to the untreated control group. FIG. 12B shows that, in response to a subsequent single stretch-unstretch maneuver that mimics a deep breath (10% magnitude, 4-sec duration), the ASM cell promptly and dramatically ablated its contraction. The contraction force gradually recovered over 180 seconds. While force ablation was similar across all three groups, the subsequent force recovery was significantly inhibited by pitavastatin treatment (* p<0.05; **** p<0.0001). All data are reported as mean and standard error of the mean (SEM).

### Example 4: Rho Kinase Inhibition

Statins inhibit the activation of Rho kinases (ROCK) in animals (A. Nohria et al., Atherosclerosis (2009) 205(2):517-21). One of the primary substrates of ROCK in regulation of actin-myosin contraction is myosin light chain 2 (MLC2) (Y. Kureishi et al., J Biol Chem (1997) 272(19):12257-60). These experiments were conducted to demonstrate that statins significantly reduce the activation of ROCK by histamine or thrombin, which in turn reduces ASM contractile forces.

Antibodies for western blot analysis against total and phospho-MLC2 were obtained from Santa Cruz Biotechnology and Cell Signaling Technology, respectively. Antibodies for pROCK1, total ROCK1, and GAPDH were obtained from Abcam. Pitavastatin was obtained from Santa Cruz Biotechnology.

Human ASM cells were treated with pitavastatin (1 μM) with or without mevalonate (MA, 200 μM) for 24 hours, then treated with histamine (10 μM) for 5 minutes. As depicted in FIG. 5, ASMs treated with pitavastatin significantly reduced histamine-induced ROCK1 phosphorylation (FIG. 5A), and this effect was abrogated by MA.

Human ASM cells were treated with pitavastatin (1 or 10 μM) for 24 hours, then treated with thrombin (2 U, 30 min). As depicted in FIG. 5B, pitavastatin (1 or 10 μM) inhibited thrombin-induced MLC2 phosphorylation.

### Example 5: Cytoskeleton Inhibition

Contractile forces in ASM are mediated by the cytoskeleton. These experiments were performed to demonstrate that statins inhibit F-actin expression, a cytoskeleton component.

Non-asthmatic primary human ASM cells were treated with either vehicle or pitavastatin (1 μM) for 24 hours. Cells were then immuno-stained for F-actin expression. As shown in FIG. 15A, pitavastatin significantly reduced basal F-actin expression. Cell lysates were analyzed by western blot for total ROCK-1, total ROCK-2, total MLC-2, and phosphorylated MLC-2. As shown in FIGS. 15C and 15D, pitavastatin reduced the total expression of ROCK-1, ROCK-2, and MLC-2 (total and phosphorylated).

Non-asthmatic primary human ASM cells were co-treated with 1 μM pitavastatin (Pit), Pit with 10 μM GGPP, or Pit with 10 μM GGPP plus 100 μM MA for 24 hrs. Pit reduced F-actin expression and ASM contraction: these reductions were abrogated by GGPP and MA (FIG. 15B).

### Example 6: Reduction/Prevention Of ASM Hypercontractility

This experiment was performed to demonstrate that statins reduce or prevent the development of airway hypercontractility independent of any anti-inflammatory effect. In this model, mice were administered nebulized methacholine (MCh) during postnatal maturation of ASM, which causes a hypercontractile phenotype without any induction of inflammatory responses.

All mouse experiments were approved by the Institutional Animal Care and Use Committee at Brigham & Women's Hospital, Harvard Medical School. The methacholine-induced hypercontractile mouse model has been described in K. R. Patel et al., FASEB J (2017) 31(10):4335-46. Briefly, mice (C57BL/6) were exposed to nebulized MCh (30 mg/mL) for 10 min daily between P15 and -20 (5 days). Control mice were administered nebulized normal saline. This establishes the MCh hyper-contractility asthma phenotype, a non-inflammatory model of asthmatic airway hyperresponsiveness AHR. Mice were administered intratracheal (i.t.) pitavastatin (5 mg/kg for five days) or vehicle control for 1 hour before each MCh nebulization. For airway % contraction assays, mouse precision-cut lung slices (PCLS) were stimulated to contract by increasing MCh concentrations (0.1-100 μM). For each measurement, at least n=4 mice were used from 2 independent experiments, and a total of 20-30 airways per lung.

Pre-treatment with intratracheal (i.t.) pitavastatin before each MCh nebulization caused a statistically significant reduction in the contraction (%) of airways (vehicle control 22.3% vs. pitavastatin 7.3%, p=0.0361, FIG. 6). This suggests pitavastatin reduces airway contraction independent of any anti-inflammatory effects. This further confirms a direct effect on the intact ASM contractile apparatus. At the molecular level, pitavastatin reduced MLC-2 phosphorylation (FIG. 5B), a crucial contractile node in regulating airway hypercontractility.

The non-inflammatory mouse model of ASM hypercontraction demonstrated that inhaled statins can target ASM to prevent a MCh-induced hypercontractile phenotype (FIG. 11B). These effects were achieved without any evidence of airway injury or toxicity (FIG. 10F). Thus, delivering pitavastatin directly to the airways via inhalation simultaneously attenuated both hallmark features of asthma-airway inflammation and ASM contraction.

### Example 7: Combination Therapies

This experiment was performed to demonstrate that statin administration directly to ASM did not interfere with the activity of β2-agonist agents.

Precision-cut human lung slices from one human donor lung were pre-treated with 5 μM Pitavastatin or vehicle (control) for 24 hours and post-treated with histamine (10 μM for 15 min) followed by isoproterenol (30 μM for an additional 30 min). The experiment was performed under serum-deprived media conditions, n=3-7 airways per group. Changes in lumen narrowing were reported as percentage changes (±SEM) of the pre-treatment state (see FIG. 13). The absolute value of lumen airway area was not statistically different between the pitavastatin and control groups at the pre-treatment state. This demonstrated that pitavastatin did not interfere with the β2-agonist effect of isoproterenol.

This experiment was performed to demonstrate that contacting ASM with a statin inhibits the release of inflammatory cytokines such as eotaxin and IL6 in response to IL13, IL17, and TNFα.

Non-asthmatic primary human ASM cells were grown to confluence and were either untreated or pre-treated with 2 mM pitavastatin and GGPP (10 mM) for a total of 72 hours. Total cytokine stimulation was for 18 hours at 10 ng/mL. As shown in FIG. 14A, pitavastatin inhibited IL13/TNFα-induced eotaxin-3 peptide secretion by a GGPP-dependent mechanism. As shown in FIG. 14B, pitavastatin also inhibited IL17/TNFα-induced IL6 peptide secretion by a GGPP-dependent mechanism. All experiments were conducted under serum-containing media conditions (10% FBS).

Normal human bronchial epithelial cells (cell line HBE1) were grown to confluence and were pre-treated with simvastatin (5 μM) and/or dexamethasone (10⁻⁷ M) for 72 hours. The cells were then treated with IL-13 (10 ng/ml). As shown in FIG. 16, each treatment independently inhibited IL13-induced eotaxin-3 extracellular secretion, and the combination of simvastatin and dexamethasone together exhibited a synergistic inhibitory effect on eotaxin-3 secretion.

FIG. 18 shows that pre-treatment with appropriate concentrations of statin potentiates the relaxation effect of dexamethasone. Primary human airway smooth muscle cells were cultured to confluence in serum-containing media (10% FBS), then pre-treated with dexamethasone (“Dex”) at 0.1, 1, or 5 μM, with or without pitavastatin (“Pit”) at 0.1, 0.5, or 1 μM concentrations for 60 hrs. The ASM cells were then exposed to a mixture of cytokines (10 n/mL IL-13, IL-17, and TNFα, “CM”) for 15 hrs, and the expression of eotaxin-3 was measured. “NT” means no treatment. Significant reductions in eotaxin-3 expression were observed when pitavastatin was added to each concentration of dexamethasone. This demonstrates that statins such as pitavastatin potentiate the therapeutic effect of dexamethasone, and indicates that inhaled statins can potentiate the therapeutic effects of inhaled corticosteroids.

This experiment was performed to demonstrate the potentiation effect of statin on β2-agonist relaxation of histamine-induced ASM contraction.

Primary ASM cells from a non-asthmatic donor were serum-deprived for 2 days in a tissue culture flask, and then cultured to confluence for an additional 24 hrs in serum-free medium in 96-well traction force measurement plates (3 kPa stiffness). The pre-treatment contractile force was measured as described above. Cells were then treated with either vehicle (PEG400), control (serum-free medium) or Pitavastatin at concentrations of 10⁻⁵ M, 10⁻⁶ M, 10⁻⁷ M, and 10⁻⁸ M, for 24 hrs, and the baseline contractile force measured. ASM cells were then acutely treated with histamine (10 μM) for 30 minutes, and the histamine-induced contractile force was measured. The cells were then treated with isoproterenol (10⁻⁶, 10⁻⁷, 10⁻⁸, 10⁻⁹, and 10⁻¹¹ M) for 30 minutes, and the isoproterenol-relaxed ASM contractile force was measured. The percent “histamine contraction” was calculated as the ratio of isoproterenol-relaxed ASM contractile force to histamine-induced contractile force. The results are shown in FIG. 17, and demonstrates that pre-treatment with appropriate concentrations of statin potentiates the relaxation effect of relevant concentrations of isoproterenol. FIG. 17A shows that at 10-11 M isoproterenol, all concentrations of statin provided essentially the same degree of relaxation, not substantially different from vehicle. However, 10⁻⁷ M, 10⁻⁶ M, and 10⁻⁵ M pitavastatin potentiated the effect of 10^{31 9} M and 10-8 M isoproterenol significantly more than vehicle. FIG. 17B details the data boxed in FIG. 17A.

### Example 8: Inhibition of Eicosanoid Mediators

Six female rhesus macaques from the Association for Assessment and Accreditation of Laboratory Animal Care (AAALAC)-accredited California National Primate Research Center (CNPRC) were used in this study. All protocols were approved by the University of California-Davis Institutional Animal Care and Use Committee and were compliant with the Animal Welfare Act and Public Health Service Policy on Humane Care and Use of Laboratory Animals. Animals were treated humanely, and care was taken to minimize and/or alleviate any pain or discomfort. The study design is shown in FIG. 7. The rhesus macaques (n=6) used in this study were age-matched at nine years and one month of age at study start and were divided into two cohorts: control and drug-treated. Simvastatin at 1 mg/kg or the simvastatin vehicle, 10% ethanol (in PBS), was delivered to the animals by aerosol mask nebulization for 40-45 minutes on 7 consecutive days. Plasma, airway epithelial cells, and bronchoalveolar lavage fluid (BALF) were sampled one day following the last exposure treatment for each phase (day=8). Tracheal and left and right lung tissue were obtained post-sacrifice five days after the end of statin exposure (day=12) (FIG. 8).

Inhaled doses of 10% ethanol were delivered to anesthetized (10 mg/kg ketamine+propofol 0.1 mg/kg/min) adult female rhesus macaques while ventilation was measured with a concurrent flow spirometry aerosol inhalation system adapted from the system described by H. C. Yeh et al., Environ Health Perspect (1976) 15:147-56.

The aerosol was generated with a jet nebulizer (MiniHEART™, Westmed, Inc., Tucson, AZ) with particle MMAD=2.5 μm and σg =2. Aerosol was conveyed through a conical clear plastic face mask with effective sealing over the nose and mouth of each animal by a flexible rubber diaphragm and a secondary seal of latex dental dam. A heated pneumotacho-graph (Model 8300A, Hans Rudolph, Inc., Kansas City, MO) connected to a pressure transducer (Model MP45-14, Validyne Engineering Corp., Northridge, CA) and computer based pulmonary physiology platform (Ponemah, DSI, Inc., St. Paul, MN) was used to measure ventilatory volume fluctuation providing real-time measurements of respiratory flows, average minute volume and total ventilation during the inhalation exposure period. Dose was estimated using aerosol concentration, estimated deposition fraction from aerodynamic size, and the total volume inhaled.

Rhesus macaques were anesthetized with ketamine (10 mg/kg), and anesthesia was maintained with propofol (0.1 mg/kg/min). As described previously (E. S. Schelegle et al., Am J Pathol (2001) 158(1):333-41), 10 mL of endotoxin-free PBS (Sigma, St. Louis, MO) was instilled through a bronchoscope. The bronchoscope was flushed three times with PBS prior to each sample to empty the channel and avoid cross-contamination. The bronchoscope and cytobrush were inserted and removed into the airways as one unit. Bronchoalveolar lavage fluid (BALF) was collected after cytobrushings were collected. Aliquots of 24 or 32 mL of PBS fluid were instilled, then withdrawn yielding ~30-50% return of BALF for the majority of samples.

BALF samples were stored on ice immediately following collection, and lavage supernatant was obtained by centrifugation for 5 min at 6,000 rpm, and then stored at −80° C. BALF was collected first from the right middle lobe, then from the left upper lobe.

Treating rhesus macaque NHP with nebulized simvastatin (1 mg/kg) inhibits two eicosanoids known to be potent airway bronchoconstrictors, leukotriene B4 (LTB4) and thromboxane B2 (TXB2). Simvastatin significantly reduced LTB4 in the BAL fluid (*p=0.0143) on day=12 (5 days after the last statin dose), but had no significant change in lung tissue (FIGS. 9A-B). Simvastatin significantly inhibited TXB2 in lung tissue (*p=0.0358), and there was a positive trend of reduced TXB2 levels in BAL fluid (p=0.051) on day=12 (FIGS. 9C-D). These results indicate that even basal levels of pro-bronchoconstricting lipid agonists are reduced in the airways of non-inflamed rhesus macaque lungs suggesting an in vivo bronchoprotective effect of inhaled statins.

On day 12, the rhesus macaques were euthanized followed by collection of their organs including the lungs. Utilizing Mass-Spectrometry based methods, the distribution of simvastatin (lactone) and its active metabolite SA were determined. Both forms were mostly present in the mainstem bronchi and the lower lobes predominantly, with relatively low levels in the gut, liver, or muscle tissues (FIG. 9). Therefore, delivering statins via inhalation appears to be a safe and feasible way of achieving high airway distribution (FIG. 10).

All publications, patents, and patent applications mentioned in this disclosure are herein incorporated by reference to the same extent as if each individual publication or patent application was specifically and individually indicated to be incorporated by reference. No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

1.-131. (canceled)

132. A method of treating chronic obstructive pulmonary disease (COPD) in a subject in need thereof, comprising administering to the subject by inhalation a therapeutically effective amount of a statin selected from the group consisting of simvastatin and pitavastatin, or an isomer, enantiomer, or diastereomer thereof.

133. The method of claim 132, wherein the statin is simvastatin.

134. The method of claim 132, wherein the statin is pitavastatin.

135. The method of claim 132, wherein the therapeutically effective amount of a statin increases airway smooth muscle (ASM) relaxation.

136. The method of claim 132, wherein the therapeutically effective amount of a statin increases stretch-induced airway smooth muscle (ASM) relaxation.

137. The method of claim 132, wherein the therapeutically effective amount of a statin reduces airway smooth muscle (ASM) contraction.

138. The method of claim 132, wherein the therapeutically effective amount of a statin reduces bronchoconstriction.

139. The method of claim 132, wherein the therapeutically effective amount of a statin reduces airway smooth muscle (ASM) release of inflammatory mediators.

140. The method of claim 132, wherein the method further comprises administering an additional therapeutic agent selected from the group consisting of a β-agonist, long-acting muscarinic antagonist, and a corticosteroid.

141. The method of claim 140, wherein the additional therapeutic agent is a β-agonist.

142. The method of claim 140, wherein the additional therapeutic agent is a corticosteroid.

143. The method of claim 140, wherein the additional therapeutic agent is selected from the group consisting of albuterol, formoterol, salbutamol, and isoproterenol.

144. The method of claim 140, wherein the additional therapeutic agent is isoproterenol.

145. The method of claim 140, wherein the additional therapeutic agent is dexamethasone.

146. The method of claim 140, wherein the additional therapeutic agent is potentiated by the statin.

147. The method of claim 140, wherein the additional therapeutic agent is administered at a sub-therapeutic dose.
